(19) United States
(12) Patent Application Publication
(10) Pub. No.: US 2005/0112571 A1
(43) Pub. Date: May 26, 2005

(54) Dna molecules conferring tolerance to herbicidal compounds

(76) Inventors: Johannes Gielen, Saint-Sauveur (FR); Fermin Azanza, Saint-Sauveur (FR)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 10/485,226

(22) PCT Filed: Aug. 1, 2002
(86) PCT No.: PCT/EP02/08593

(30) Foreign Application Priority Data
Aug. 2, 2001 (GB) 0118928.1

(51) Int. Cl. A01H 1/00; C12N 15/82; C12N 5/04; C12Q 1/68; C07H 21/04

(52) U.S. Cl. 435/6; 435/419; 435/468; 800/278; 536/23.2

(57) ABSTRACT

The present invention relates to tolerance to inhibitors of ALS/AHAS activity in plants, in particular to DNA molecules encoding herbicide tolerant proteins having ALS/AHAS activity. The present invention also relates to molecular markers used to detect tolerance to the herbicide and to methods using such molecular markers.

The present invention relates to the field of herbicide tolerance, in particular to DNA molecules conferring tolerance to herbicidal compounds and to molecular markers to detect herbicide tolerance.

The use of herbicides to control undesirable vegetation such as weeds in crop fields has become almost a universal practice. The herbicide market exceeds 15 billion dollars annually. Despite this extensive use, weed control remains a significant and costly problem for farmers. New weeds regularly appear in cultured fields, some of which are particularly difficult to control. Herbicides that exhibit greater potency, broader weed spectrum, and more rapid degradation in soil can also, unfortunately, have greater crop phytotoxicity.

One solution applied to this problem has been to use crops that are resistant or tolerant to herbicides. Crop hybrids or lines tolerant to the herbicides allow for the use of the herbicides to kill weeds without attendant risk of damage to the crop. Development of tolerance can allow application of an herbicide to a crop where its use was previously precluded or limited (e.g. to pre-emergence use) due to sensitivity of the crop to the herbicide.

Recently, there have been reports of wild sunflower tolerant to the herbicide imazethapyr, an imidazolinone-type herbicide (Al-Khatib et al, 1998. Weed Science 46: 403). The use of this tolerance has been described for post-emergence application of imazethapyr on sunflower to control the parasitic weed broomrape, a growing problem in Southern and Eastern Europe (EP 1088480). Thus, herbicide tolerance appears to be a promising weed control strategy in sunflower. However, the molecular basis for the tolerance mechanism remains unknown, preventing the broad application of this new tolerance mechanism in agriculture. Accordingly, there is an unfulfilled need to understand the molecular basis of this herbicide tolerance.

Moreover, because of the absence of knowledge on the molecular basis of the tolerance to the herbicide, it is difficult to follow the trait during the various stages of breeding for new lines, during seeds production and to assure the purity of commercial seeds lots. Selection of plants tolerant to the herbicide can only be achieved by application of the herbicide, which requires planting seeds on large acreages and spraying plants with high amounts of the herbicide. This is expensive and time consuming, delaying the market introduction of much wanted tolerant lines.

Therefore, there is also an unfulfilled need for new and inexpensive ways to follow and assess the presence of the tolerance trait, in particular in sunflower.

The present invention therefore addresses the need to understand and dissect new mechanisms of herbicide tolerance in plants and to apply such herbicide tolerance to a wide variety of crops. The present invention also addresses the need for improved ways to detect and follow the herbicide tolerance in plants.

Accordingly, in one aspect, the present invention discloses for the first time the molecular basis for the tolerance to the herbicide imazethapyr in sunflower. The inventors of the present invention have determined that an amino acid substitution (Y445H) in the sunflower ALS/AHAS protein underlies the tolerance to imazethapyr. Unexpectedly, this amino acid substitution is novel and different from other mutations conferring tolerance to inhibitors of ALS/AHAS activity, which were previously known in other plant species, such as Arabidopsis or maize (see for example U.S. Pat. Nos. 6,225,105 and 5,767,361; Wright et al, 1998, Weed Sci 46: 13-23). The present invention therefore discloses a DNA molecule comprising a nucleotide sequence encoding a protein having ALS/AHAS activity comprising the amino acid substitution described in the present invention, wherein the amino acid substitution confers tolerance to an inhibitor of ALS/AHAS activity. The inventors of the present invention have also isolated and determined the nucleotide sequence and amino acid sequence of the acetolactate synthase (ALS)/acetohydroxyacid synthase (AHAS) of sunflower (Helianthus annuus).

In another aspect, the present invention also discloses molecular markers for the detection of the tolerance trait. The inventors of the instant invention have determined sequence divergences or polymorphisms between an herbicide tolerant allele and a series of susceptible alleles, and have exploited this feature to develop molecular markers associated with the tolerance. In particular, the inventors have developed co-dominant markers, which are particularly useful in differentiating between plants or lines heterozygous or homozygous for the herbicide tolerance trait.

In a further aspect, the present invention also provides methods for mapping the tolerance trait in tolerant plants, for detecting the presence of an herbicide tolerant allele in a plant, and for transferring the tolerance trait to a susceptible or less tolerant plant. The present invention also discloses methods to control the quality of a lot of herbicide tolerant seeds.

The DNA molecules disclosed in the instant invention are used to confer tolerance to inhibitors of ALS/AHAS activity in a wide variety of crops and thus provide much desired alternative strategies for herbicide tolerance. Moreover, the markers and methods disclosed herein offer a clear advantage over previously known methods. They represent cheaper and faster alternatives for the introgression of the tolerance trait and for quality control. Such markers and methods allow for quicker release of herbicide tolerant lines to the market and for a better quality control of commercial seeds lots.

The present invention therefore discloses:

An isolated DNA molecule comprising a nucleotide sequence that encodes a protein having acetolactate synthase (ALS)/acetohydroxyacid synthase (AHAS) activity, wherein said protein comprises an amino acid sequence set forth in SEQ ID NO: 2. In a preferred embodiment, the DNA molecule comprises a nucleotide sequence set forth in SEQ ID NO:1. In another preferred embodiment, the plant nucleotide sequence encodes a sunflower protein having ALS/AHAS activity.

The present invention further discloses:

An isolated DNA molecule comprising a nucleotide sequence that encodes a protein having ALS/AHAS activity, wherein said protein comprises an amino acid substitution occurring at a position corresponding to position 475 in the comparative alignment shown in Table 3, wherein said amino acid substitution confers to said protein tolerance to an inhibitor of a protein having ALS/AHAS activity which does not comprise said amino acid substitution. In a preferred embodiment, the amino acid substitution occurs at a position corresponding to position 445 in the amino acid sequence set forth in SEQ ID NO:4. In another preferred embodiment, a tyrosine is replaced by a histidine, i.e. a tyrosine corresponding to position 475 in the comparative alignment shown in Table 3 is replaced by a histidine. Preferably, the amino acid substitution confers to said protein tolerance to an imidazolinone herbicide. In a preferred embodiment, the nucleotide sequence encodes a plant protein having ALS/AHAS activity, preferably from a dicotyledonous plant. In another preferred embodiment, the nucleotide sequence encodes a protein having ALS/AHAS activity from a monocotyledonous plant. In another preferred embodiment, the nucleotide sequence encodes a sunflower protein having ALS/AHAS activity. In another preferred embodiment, the amino acid sequence of said protein is set forth in SEQ ID NO:4.

In another preferred embodiment, the nucleotide sequence comprises a nucleotide substitution at a position corresponding to any one of positions 1566 or 1567 in SEQ ID NO:1. In another preferred embodiment, a nucleotide at a position corresponding to position 1566 in SEQ ID NO:1 is a cytosine. In yet another preferred embodiment, the nucleotide sequence that encodes a protein having ALS/AHAS activity further comprises a nucleotide substitution at any one of positions 1229, 1232 or 1235 in SEQ ID NO:1. Preferably, a nucleotide at a position corresponding to position 1229 in SEQ ID NO:1 is a guanine, a nucleotide at a position corresponding to position 1232 in SEQ ID NO:1 is a thymine and a nucleotide at a position corresponding to position 1235 in SEQ ID NO:1 is a cytosine. Preferably, the nucleotide sequence that encodes a protein having ALS/AHAS activity is set forth in SEQ ID NO:3.

The present invention further discloses:

A chimeric gene comprising a DNA molecule disclosed above operatively linked to a promoter functional in a host cell. A recombinant vector comprising a DNA molecule disclosed above operatively linked to a promoter functional in a host cell. A host cell comprising a DNA molecule disclosed above operatively linked to a promoter functional in the host cell.

Preferably, the host cell is a plant cell or a bacterial cell.

The present invention further discloses:

A transgenic plant, plant tissue or plant cell comprising a DNA molecule disclosed above operatively linked to a promoter functional in the plant, plant tissue or plant cell, wherein the nucleotide sequence is expressed in the plant, plant tissue or plant cell and confers to the plant, plant tissue or plant cell tolerance to an inhibitor of ALS/AHAS activity. Preferably, the inhibitor of ALS/AHAS activity is an imidazolinone herbicide.

The present invention further discloses:

Seed of a plant disclosed above, including seed of the progeny of the plant, wherein the seed comprises the DNA molecule.

The present invention further discloses:

A method for controlling the growth of undesired vegetation, which comprises applying to a population of a plant disclosed above an effective amount of an inhibitor of ALS/AHAS activity. Preferably, the inhibitor of ALS/AHAS activity is an imidazolinone herbicide.

Preferably, the plant is selected from the group consisting of sunflower, sugar cane, soybean, barley, cotton, tobacco, sugar beet, oilseed rape, maize, wheat, sorghum, rye, oats, turf and forage grasses, millet, forage and rice.

The present invention further discloses:

An isolated protein having ALS/AHAS activity, wherein the protein comprises the amino acid sequence set forth in SEQ ID NO:2.

An isolated protein having ALS/AHAS activity, wherein the protein comprises an amino acid substitution occurring at a position corresponding to position 475 in the comparative alignment shown in Table 3, wherein the amino acid substitution confers to the protein tolerance to an inhibitor a protein having ALS/AHAS activity which does not comprise the amino acid substitution. In a preferred embodiment, the amino acid substitution occurs at a position corresponding to position 445 in the amino acid sequence set forth in SEQ ID NO:4. In another preferred embodiment, a tyrosine is replaced by a histidine, i.e. a tyrosine corresponding to position 475 in the comparative alignment shown in Table 3 is replaced by a histidine. Preferably, the amino acid substitution confers to the protein tolerance to an imidazolinone herbicide. In a preferred embodiment, the protein having ALS/AHAS activity is a plant protein, preferably from a dicotyledonous plant. In another preferred embodiment, the protein having ALS/AHAS activity is from a monocotyledonous plant. In another preferred embodiment, the protein is a sunflower protein. In another preferred embodiment, the protein comprises the amino acid sequence set forth in SEQ ID NO:4.

The present invention further discloses:

An isolated DNA molecule comprising about 15 successive nucleotides of the nucleotide sequence set forth in SEQ ID NO:1 or SEQ ID NO:3, wherein the DNA molecule comprises a nucleotide polymorphism, wherein the polymorphism allows to differentiate between an allele conferring tolerance to an inhibitor of ALS/AHAS activity and an allele sensitive to the inhibitor, preferably in a sunflower plant. Preferably, the polymorphism allows to differentiate between a nucleotide sequence encoding an imidazolinone-sensitive ALS/AHAS and a nucleotide sequence encoding an imidazolinone-tolerant ALS/AHAS in a sunflower plant. Preferably, DNA molecule comprises about 20 successive nucleotides of the nucleotide sequence set forth in SEQ ID NO:1 or SEQ ID NO:3. Preferably, DNA molecule comprises about 50 successive nucleotides of the nucleotide sequence set forth in SEQ ID NO:1 or SEQ ID NO:3. In another preferred embodiment, the polymorphism is at a position corresponding to any one of positions 1566, 1229, 1232 or 1235 in SEQ ID NO:1. In a preferred embodiment, the polymorphism is at a position corresponding to position 1566 in SEQ ID NO:1. Preferably, the DNA molecule comprises any one of the nucleotide sequences set forth in SEQ ID NO:8 (HiNK379) or SEQ ID NO:9 (HiNK415). In an alternative preferred embodiment, the polymorphism is at a position corresponding to any one of positions 1229, 1232 or 1235 in SEQ ID NO:1. Preferably, the DNA molecule further comprises any one of SEQ ID NO: 10 (HiNK451), SEQ ID NO: 12 (HiNK414), SEQ ID NO:11 (HiNK452) or SEQ ID NO:13 (HiNK415). In another preferred embodiment, the DNA molecule further comprises any one of SEQ ID NO:14 (HiNK702), SEQ ID NO:15 (HiNK703), SEQ ID NO:16 (HiNK700) or SEQ ID NO:17 (HiNK701). In yet another preferred embodiment, the DNA molecule is an amplified PCR fragment. In yet another preferred embodiment, the DNA molecule is from about 100 bp to about 3,000 bp long, preferably from about 200 bp to about 2,000 bp long, more preferably about 500 to 1,500 bp long.

The present invention further discloses:

An isolated DNA molecule comprising about 15 successive nucleotides of a nucleotide sequence encoding a protein having ALS/AHAS activity, wherein the DNA molecule comprises a nucleotide polymorphism at a position corresponding to any one of positions 1566-1568 in SEQ ID NO:1, wherein the polymorphism allows to differentiate between an allele conferring tolerance to an inhibitor of ALS/AHAS activity and an allele sensitive to the inhibitor. Preferably, DNA molecule comprises about 20 successive nucleotides of a nucleotide sequence encoding a protein having ALS/AHAS activity, preferably about 50 successive nucleotides of the nucleotide sequence. In another preferred embodiment, the polymorphism is at a position corresponding to position 1566 in SEQ ID NO:1. In another preferred embodiment, the DNA molecule is an amplified PCR fragment. In yet another preferred embodiment, the DNA molecule is from about 100 bp to about 3,000 bp long, preferably from about 200 bp to about 2,000 bp long, more preferably about 500 to 1,500 bp long.

The present invention further discloses:

An oligonucleotide capable of hybridizing to a DNA molecule above, wherein the oligonucleotide comprises a nucleotide corresponding to any one of positions 1229, 1232 or 1235 or 1566 of SEQ ID NO:1 or a complement thereto. Preferably, the oligonucleotide further comprises a detectable label. Preferably, the oligonucleotide is from about 8 bp to about 50 bp long, preferably from about 10 bp to about 30 bp long, preferably from about 15 to 25 nt long.

The present invention further discloses:

A method of identifying a plant tolerant to an inhibitor of ALS/AHAS activity comprising the steps of: a) obtaining a sample from a plant; b) detecting in the sample a DNA molecule disclosed above, the presence of the DNA molecule being indicative of an allele conferring tolerance to an inhibitor of ALS/AHAS activity in the plant, wherein the plant is tolerant to an inhibitor of ALS/AHAS activity. Preferably, the plant is a sunflower plant.

A method of selecting a plant tolerant to an inhibitor of ALS/AHAS activity from a population of plants comprising the steps of: a) providing a population of plants; b) obtaining a sample of a plant of the population; c) detecting in the sample a DNA molecule disclosed above, the presence of the DNA molecule being indicative of an allele conferring tolerance to an inhibitor of ALS/AHAS activity in the plant; d) selecting the plant, wherein the plant is tolerant to an inhibitor of ALS/AHAS activity. Preferably, the plant is a sunflower plant.

A method for introgressing tolerance to an inhibitor of ALS/AHAS activity into a plant comprising the steps of: a) obtaining a plant tolerant to inhibitor of ALS/AHAS activity; b) crossing the plant of step a) with a plant which is sensitive or less tolerant to the inhibitor; c) detecting in a plant resulting from the cross in step b) a DNA molecule disclosed above, the presence of the DNA molecule being indicative of an allele conferring tolerance to an inhibitor of ALS/AHAS activity in the plant; d) selecting a plant of step c) for further breeding, wherein the plant is tolerant to an inhibitor of ALS/AHAS activity. Preferably, the methods further comprises repeating steps b) to d) until the tolerance to the inhibitor is introgressed into the plant which sensitive or less tolerant to the inhibitor. Preferably, the plant is a sunflower plant. Preferably, in any one of the methods disclosed above, the inhibitor of ALS/AHAS activity is an imidazolinone herbicide.

The present invention further discloses:

A kit for detecting a single nucleotide polymorphism indicative for tolerance or sensitivity to an inhibitor of ALS/AHAS activity in a plant comprising an oligonucleotide disclosed above. Preferably, the kit comprises any one of oligonucleotides set forth in SEQ ID NO:8 or SEQ ID NO:9. In another preferred embodiment, the kit comprises any one of oligonucleotides set forth in SEQ ID NO:10, SEQ ID NO:12, SEQ ID NO:11 or SEQ ID NO:13. In yet another preferred embodiment, the kit comprises any one of oligonucleotides SEQ ID NO:14, SEQ ID NO:15, SEQ ID NO:16 or SEQ ID NO:17.

The present invention further discloses:

Use of a DNA molecule disclosed above to identify a plant tolerant to an inhibitor of ALS/AHAS activity. Preferably, the plant is a sunflower plant.

Use of a DNA molecule disclosed above to confer to a plant tolerance to an inhibitor of ALS/AHAS activity.

The present invention further discloses:

A method for obtaining a plant, plant cell or plant tissue tolerant to an inhibitor of ALS/AHAS activity, wherein the tolerance is due to an amino acid substitution of the present invention in a protein having ALS/AHAS activity of the plant, plant cell or plant tissue. In a preferred embodiment, the method comprises introducing into the plant, plant cell or plant tissue a DNA molecule comprising a nucleotide sequence encoding herbicide-tolerant plant of the present invention, wherein the plant, plant cell or plant tissue is tolerant to the inhibitor. In another preferred embodiment, the method comprises introducing a nucleotide substitution of the present invention in a nucleotide sequence of the plant, plant cell or plant tissue which encodes a protein having ALS/AHAS activity. Preferably, the nucleotide substitution is introduced in the nucleotide sequence by mutagenesis, preferably by chemical or physical mutagenesis, or by homologous recombination or oligonucleotide-based mutagenesis. In another preferred embodiment, the plant, plant cell or plant tissue is selected for tolerance to an imidazolinone.

The present invention further discloses:

A method to determine whether a plant is homozygous or heterozygous for an allele conferring tolerance to an inhibitor of ALS/AHAS activity comprising: a) obtaining a sample of a plant; b) detecting in the sample a DNA molecule disclosed above, wherein the step of detecting the DNA molecule is carried out using a co-dominant marker; c) determining whether a nucleotide sequence encoding a protein having ALS/AHAS activity tolerant to an inhibitor of ALS/AHAS activity is heterozygous or homozygous in the plant. Preferably, the plant is a sunflower plant.

### Definitions

Allele: one of several alternate forms of a nucleotide sequence occupying a given locus on a chromosome. In different alleles, the alternate forms of the nucleotide sequence may for example encode alternate forms of a protein.

Co-dominant: when referring to a molecular marker, a co-dominant marker allows for the detection of two different alleles, for example in a plant heterozygous for the allele. In the present invention, a co-dominant marker allows for the detection of an herbicide-tolerant allele and an herbicide-sensitive allele in a plant heterozygous for the herbicide-tolerant allele. Corresponding to: in the context of the present invention, “corresponding to” means that when nucleotide or amino acid sequences are aligned with each other, such as in Tables 1-3, the nucleotides or amino acids that “correspond to” certain enumerated positions in a Table are those that align with these positions in the Table, but that are not necessarily in these exact numerical positions relative to the particular nucleotide or amino acid sequence. Likewise, when a particular nucleotide or amino acid sequence is aligned with a reference nucleotide or amino acid sequence (for example, a sunflower sequence given in SEQ ID NO:1-4), the nucleotides or amino acids of the particular sequence that “correspond to” certain enumerated positions of the reference sequence are those that align with these positions of the reference sequence, but are not necessarily in these exact numerical positions of the particular nucleotide or amino acid sequence.

Enzyme activity: means herein the ability of an enzyme to catalyze the conversion of a substrate into a product. A substrate for the enzyme comprises the natural substrate of the enzyme but also comprises analogues of the natural substrate, which can also be converted by the enzyme into a product or into an analogue of a product. The activity of the enzyme is measured for example by determining the amount of product in the reaction after a certain period of time, or by determining the amount of substrate remaining in the reaction mixture after a certain period of time. The activity of the enzyme is also measured by determining the amount of an unused co-factor of the reaction remaining in the reaction mixture after a certain period of time or by determining the amount of used co-factor in the reaction mixture after a certain period of time. The activity of the enzyme is also measured by determining the amount of a donor of free energy or energy-rich molecule (e.g. ATP, phosphoenolpyruvate, acetyl phosphate or phosphocreatine) remaining in the reaction mixture after a certain period of time or by determining the amount of a used donor of free energy or energy-rich molecule (e.g. ADP, pyruvate, acetate or creatine) in the reaction mixture after a certain period of time. In the context of the present invention, a preferred enzyme activity is the ALS/AHAS activity, which is carried out by a protein having ALS/AHAS activity. A protein having ALS/AHAS activity comprises an ALS/AHAS protein or a functional fragment or mutant of the ALS/AHAS protein, wherein the fragment or mutant is capable of carrying out the ALS/AHAS activity. The ALS/AHAS activity can be determined by various methods known in the art, and described for example in Chaleff et al., Science 224:1443-1445 (1984), and as modified by Haughn et al., Plant Physiol. 92:1081-1085 (1988) and by Singh et al., Analy. Biochem. 171:1173-179 (1988), or in U.S. Pat. Nos. 6,225,105 and 5,767,361.

Herbicide: a chemical substance used to kill or suppress the growth of plants, plant cells, plant seeds, or plant tissues. In the context of the present invention, a preferred herbicide is a chemical substance that inhibits the activity of a protein having ALS/AHAS activity.

Heterologous DNA Sequence: a DNA sequence not naturally associated with a host cell into which it is introduced, including non-naturally occurring multiple copies of a naturally occurring DNA sequence; and genetic constructs wherein an otherwise homologous DNA sequence is operatively linked to a non-native sequence.

Homologous DNA Sequence: a DNA sequence naturally associated with a host cell into which it is introduced.

Inhibitor: a chemical substance that causes abnormal growth, e.g., by inactivating the enzymatic activity of a protein such as a biosynthetic enzyme, receptor, signal transduction protein, structural gene product, or transport protein that is essential to the growth or survival of the plant. In the context of the instant invention, an inhibitor is a chemical substance that alters the enzymatic activity of a protein having ALS/AHAS activity. More generally, an inhibitor causes abnormal growth of a host cell by interacting with a protein having ALS/AHAS activity.

Isogenic: plants which are genetically identical, except that they may differ by the presence or absence of a heterologous DNA sequence.

Isolated: in the context of the present invention, an isolated DNA molecule or an isolated enzyme is a DNA molecule or enzyme that, by the hand of man, exists apart from its native environment and is therefore not a product of nature. An isolated DNA molecule or enzyme may exist in a purified form or may exist in a non-native environment such as, for example, in a transgenic host cell.

Marker assisted selection: refers to the process of selecting a desired trait or desired traits in a plant or plants by detecting one or more nucleic acids from the plant, where the nucleic acid is associated with the desired trait.

Mature protein: protein which is normally targeted to a cellular organelle, such as a chloroplast, and from which the transit peptide has been removed.

Minimal Promoter: promoter elements, particularly a TATA element, that are inactive or that have greatly reduced promoter activity in the absence of upstream activation. In the presence of a suitable transcription factor, the minimal promoter functions to permit transcription.

Modified Enzyme Activity: enzyme activity different from that which naturally occurs in a plant (i.e. enzyme activity that occurs naturally in the absence of direct or indirect manipulation of such activity by man), which is tolerant to inhibitors that inhibit the naturally occurring enzyme activity.

Pre-protein: protein which is normally targeted to a cellular organelle, such as a chloroplast, and still comprising its transit peptide.

Significant Increase: an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, and most preferably an increase by about 10-fold or greater.

Significantly less: means that the amount of a product of an enzymatic reaction is reduced by more than the margin of error inherent in the measurement technique, preferably a decrease by about 2-fold or greater of the activity of the wild-type enzyme in the absence of the inhibitor, more preferably an decrease by about 5-fold or greater, and most preferably an decrease by about 10-fold or greater.

Single nucleotide polymorphism (or “SNP”) or polymorphism: used herein to describe any nucleotide sequence variation. Preferably, such a variation is common in a population of organisms and is inherited in a Mendelian fashion. Such alleles may or may not have associated phenotypes.

Substantially similar: with respect to a gene of the present invention, in its broadest sense, the term “substantially similar”, when used herein with respect to a nucleotide sequence, means a nucleotide sequence corresponding to a reference nucleotide sequence, wherein the corresponding sequence encodes a polypeptide having substantially the same structure and function as the polypeptide encoded by the reference nucleotide sequence, e.g. where only changes in amino acids not affecting the polypeptide function occur. Desirably the substantially similar nucleotide sequence encodes the polypeptide encoded by the reference nucleotide sequence. The term “substantially similar” is specifically intended to include nucleotide sequences wherein the sequence has been modified to optimize expression in particular cells. A nucleotide sequence “substantially similar” to reference nucleotide sequence hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

With respect to a protein of the present invention, the term “substantially similar”, when used herein with respect to a protein, means a protein corresponding to a reference protein, wherein the protein has substantially the same structure and function as the reference protein, e.g. where only changes in amino acids sequence not affecting the polypeptide function occur.

One skilled in the art is also familiar with analysis tools, such as GAP analysis, to determine the percentage of identity between the “substantially similar” and the reference nucleotide sequence, or protein or amino acid sequence. In the present invention, “substantially similar” is therefore also determined using default GAP analysis parameters with the University of Wisconsin GCG, SEQWEB application of GAP, based on the algorithm of Needleman and Wunsch (Needleman and Wunsch (1970) J. Mol. Biol. 48: 443-453).

Substrate: a substrate is the molecule that an enzyme naturally recognizes and converts to a product in the biochemical pathway in which the enzyme naturally carries out its function, or is a modified version of the molecule, which is also recognized by the enzyme and is converted by the enzyme to a product in an enzymatic reaction similar to the naturally-occurring reaction.

Tolerance: the ability to continue essentially normal growth or function when exposed to an inhibitor or herbicide in an amount sufficient to suppress the normal growth or function of native, unmodified plants or enzymes.

Transformation: a process for introducing heterologous DNA into a cell, tissue, or plant.

Transformed cells, tissues, or plants are understood to encompass not only the end product of a transformation process, but also transgenic progeny thereof.

Transgenic: stably transformed with a recombinant DNA molecule that preferably comprises a suitable promoter operatively linked to a DNA sequence of interest.

### BRIEF DESCRIPTION OF THE SEQUENCE LISTING

SEQ ID NO:1 nucleotide sequence encoding a protein having ALS/AHAS activity
SEQ ID NO:2 amino acid sequence encoded by the nucleotide sequence of SEQ ID NO:1
SEQ ID NO:3 nucleotide sequence encoding an herbicide-tolerant protein having ALS/AHAS activity
SEQ ID NO:4 amino acid sequence encoded by the nucleotide sequence of SEQ ID NO:3
SEQ ID NO:5 Oligonucleotide HiNK366
SEQ ID NO:6 Oligonucleotide HiNK369
SEQ ID NO:7 Oligonucleotide HiNK370
SEQ ID NO:8 Oligonucleotide HiNK379
SEQ ID NO:9 Oligonucleotide HiNK415
SEQ ID NO:10 Oligonucleotide HiNK451
SEQ ID NO:11 Oligonucleotide HiNK414
SEQ ID NO:12 Oligonucleotide HiNK452
SEQ ID NO:13 Oligonucleotide HiNK415
SEQ ID NO:14 Oligonucleotide HiNK702
SEQ ID NO:15 Oligonucleotide HiNK703
SEQ ID NO:16 Oligonucleotide HiNK700
SEQ ID NO:17 Oligonucleotide HiNK701

The present invention discloses a new mechanism of tolerance to inhibitors of ALS/AHAS activity in plants. The inventors of the present invention have determined that a novel amino acid substitution in the acetolactate synthase (ALS) or acetohydroxyacid synthase (AHAS) protein underlies the tolerance to an inhibitor. The inventors of the present invention have also determined the nucleotide and amino acid sequence of a sunflower ALS/AHAS. The DNA molecules of the present invention are used to confer tolerance to inhibitors of ALS/AHAS activity in a wide variety of crops. Molecular markers based on these DNA molecules and on polymorphisms between herbicide tolerant and herbicide sensitive alleles are also disclosed. These markers are used in breeding new varieties tolerant to herbicides and in seeds production.

The ALS/AHAS catalyzes the first common step in the biosynthetic pathway of the amino acids valine, leucine and isoleucine, and is known under both names ALS and AHAS (EC 4.1.3.18). ALS/AHAS is the target for various classes or herbicidal compounds, such as the imidazolinones, sulfonylureas and triazolopyrimidines. Mutations conferring tolerance to such inhibitors of ALS/AHAS activity in plants have been described in several crops, but unexpectedly, the inventors of the present invention disclose here a novel mutation conferring tolerance to inhibitors of ALS/AHAS activity.

Accordingly, in one aspect, the inventors of the present invention disclose herein the isolation and identification of the nucleotide sequence and amino acid sequence of an acetolactate synthase (ALS) or acetohydroxyacid synthase (AHAS) from sunflower (Helianthus annuus).

The nucleotide sequence encoding the sunflower ALS/AHAS is set forth in SEQ ID NO:1, and the corresponding amino acid sequence is set forth in SEQ ID NO:2. The procedure leading to isolation of the nucleotide and amino acid sequence of the sunflower ALS/AHAS are described in example 2.

A nucleotide sequence encoding a sunflower ALS/AHAS is for example expressed in a transgenic plant to confer tolerance to said plant to an inhibitor of the ALS/AHAS activity naturally occurring in said plant. According to this embodiment, plants, plant tissue, plant seeds, or plant cells are transformed, preferably stably transformed, with a recombinant DNA molecule comprising a suitable promoter functional in plants operatively linked to a nucleotide sequence encoding a sunflower ALS/AHAS. The transgenic plants, plant tissue, plant seeds, or plant cells thus created are then selected using conventional techniques disclosed herein or well-known in the art, whereby herbicide tolerant lines are isolated, characterized, and developed. Increased expression of the nucleotide sequence results in a level of ALS/AHAS activity at least sufficient to overcome growth inhibition caused by an herbicide when applied in amounts sufficient to inhibit normal growth of control plants. The level of expressed protein generally is at least two times, preferably at least five times, and more preferably at least ten times the natively expressed amount.

A nucleotide sequence encoding a sunflower ALS/AHAS is also expressed recombinantly in a host cell and used to screen for chemicals that selectively inhibit a sunflower ALS/AHAS.

Suitable expression vectors and methods for recombinant production of proteins are well known for host organisms such as E. coli, yeast, and insect cells (see, e.g., Luckow and Summers, Bio/Technol. 6: 47 (1988), and baculovirus expression vectors, e.g., those derived from the genome of Autographica californica nuclear polyhedrosis virus (AcMNPV). A preferred baculoviris/insect system is pAcHLT (Pharmingen, San Diego, Calif.) used to transfect Spodoptera frugiperda Sf9 cells (ATCC) in the presence of linear Autographa californica baculovirus DNA (Pharmigen, San Diego, Calif.). The resulting virus is used to infect HighFive Tricoplusia ni cells (Invitrogen, La Jolla, Calif.). Recombinantly produced protein is isolated and purified using a lines of standard techniques. The actual techniques that may be used will vary depending upon the host organism used, whether the protein is designed for secretion, and other such factors familiar to the skilled artisan (see, e.g. chapter 16 of Ausubel, F. et al., “Current Protocols in Molecular Biology”, pub. by John Wiley & Sons, Inc. (1994)).

Accordingly, the present invention also discloses recombinant DNA molecules, expression cassettes and recombinant vectors comprising a nucleotide sequence encoding a sunflower ALS/AHAS. The present invention also discloses cells, e.g. plant, bacterial and insect cells, plant tissue and plants including the seeds and progeny thereof comprising such nucleotide sequences, which are preferably tolerant to an inhibitor of ALS/AHAS activity.

The present invention also discloses an amino acid substitution in the amino acid sequence of a protein having ALS/AHAS activity, wherein the amino acid substitution confers tolerance to an inhibitor of a corresponding protein which does not comprise the amino acid substitution. The amino acid substitution is at a position corresponding to position 475 in the comparative alignment shown in Table 3. Preferably, a tyrosine (Y) at said position is replaced by a histidine (H). Preferably, a tyrosine at a position corresponding to position 475 in Table 3 is substituted in a plant protein having ALS/AHAS activity, and confers to said protein tolerance to the inhibitor.

Thus, the present invention discloses an isolated DNA molecule comprising a nucleotide sequence that encodes a protein having ALS/AHAS activity, wherein said protein comprises an amino acid substitution occurring at a position corresponding to position 475 in the comparative alignment shown in Table 3, wherein said amino acid substitution confers to said protein tolerance to an inhibitor of a protein having ALS/AHAS activity which does not comprise said amino acid substitution.

In another preferred embodiment, the present invention discloses an isolated DNA molecule comprising a nucleotide sequence that encodes a protein having ALS/AHAS activity, wherein the protein comprises any one of the amino acid sub-sequences PQΔ₁AI, PΔ₁AI or PQΔ₁A, wherein Δ₁ is an amino acid other than tyrosine. Preferably, Δ₁ is histidine.

A preferred protein having ALS/AHAS activity of the present invention is an ALS/AHAS protein from a dicotyledonous plant or from a monocotyledonous plant. Preferably, the ALS/AHAS protein is from Amaranthus sp., Arabidopsis thaliana, Bassia scoparia, Brassica napus, Gossypium hirsutum, Nicotiana tabacum, maize, rice or xanthium. The amino acid sequences of the ALS/AHAS proteins of these crops are shown at Table 3.

Also encompassed in the present invention are nucleotide sequences encoding herbicide-tolerant proteins having ALS/AHAS activity of the present invention. In a preferred embodiment, a codon encoding a substituted amino acid is at a position corresponding to positions 1415-1417 in Table 1 or corresponding to positions 1566-1568 in SEQ ID NO:1. Preferably, a triplet “TAT” or TAC” is substituted, preferably to a “CAT” or “CAC” triplet, coding for a histidine. One skilled in the art has no difficulties to find and modify the corresponding codons in further nucleotide sequences encoding proteins having ALS/AHAS activity, so that they encode an herbicide-tolerant protein.

A particularly preferred tolerant protein having ALS/AHAS activity of the present invention is a sunflower ALS/AHAS protein. The position of the amino acid substitution corresponds to position 445 in SEQ ID NO:2. Preferably, the tolerant sunflower ALS/AHAS protein comprises the amino acid sequence set forth in SEQ ID NO:4. A preferred nucleotide sequence encoding such amino acid sequence comprises the nucleotide sequence set forth in SEQ ID NO:1 with a “T” to “C” substitution at position 1566 of SEQ ID NO:1. A particularly preferred nucleotide sequence encoding a tolerant sunflower ALS/AHAS protein is set forth in SEQ ID NO:3.

Nucleotide sequences encoding a tolerant sunflower ALS/AHAS protein are used to confer tolerance to inhibitors of naturally-occurring ALS/AHAS activity. In a preferred embodiment, such nucleotide sequences are transformed into plants or plant cells using methods and techniques disclosed herein or well-known in the art. Accordingly, the present invention also discloses recombinant DNA molecules, expression cassettes and recombinant vectors comprising a nucleotide sequence encoding an herbicide-tolerant protein having ALS/AHAS activity of the present invention. The present invention also discloses cells, e.g. plant, bacterial and insect cells, plant tissue and plants, including the seeds and progeny thereof, comprising such nucleotide sequence. The transgenic plants, plant tissue, plant seeds, or plant cells thus created are then selected using conventional techniques disclosed herein or well-known in the art, whereby herbicide tolerant lines are isolated, characterized, and developed. Expression of the nucleotide sequence results in tolerance at least sufficient to overcome growth inhibition caused by an herbicide when applied in amounts sufficient to inhibit normal growth of control plants.

In another preferred embodiment, an herbicide-tolerant allele encoding an herbicide-tolerant ALS/AHAS protein of the present invention is obtained by direct selection in plants, plant cells or plant tissue. Determination of the lowest dose of herbicide used in the selection experiments is routine in the art. Mutagenesis of plant material is preferably utilized to increase the frequency at which tolerant alleles occur in the selected population. Mutagenized seed material is derived from a variety of sources, including chemical or physical mutagenesis of seeds or pollen (Neuffer, In Maize for Biological Research Sheridan, ed. Univ. Press, Grand Forks, N.Dak., pp. 61-64 (1982)), which is then used to fertilize plants and the resulting M₁ mutant seeds collected. Preferred chemical agents for mutagenesis include ethyl methane sulfonate or methyl methane sulfonate. Preferred physical agents for mutagenesis include gamma rays, UV light or fast neutrons. Typically for Arabidopsis, M₂ seeds (Lehle Seeds, Tucson, Ariz.), which are progeny seeds of plants grown from seeds mutagenized with chemicals or with physical agentsare plated at densities of up to 10,000 seeds/plate (10 cm diameter) on minimal salts medium containing an appropriate concentration of inhibitor to select for tolerance. Seedlings that continue to grow and remain green 7-21 days after plating are transplanted to soil and grown to maturity and seed set. Progeny of these seeds are tested for tolerance to the herbicide. If the tolerance trait is dominant, plants whose seed segregate 3:1/tolerant:sensitive are presumed to have been heterozygous for the tolerance at the M₂ generation. Plants that give rise to all tolerant seed are presumed to have been homozygous for the resistance at the M₂ generation. Such mutagenesis on intact seeds and screening of their M2 progeny seed can also be carried out on other species, for instance soybean (see, e.g. U.S. Pat. No. 5,084,082). Alternatively, mutant seeds to be screened for herbicide tolerance are obtained as a result of fertilization with pollen mutagenized by chemical or physical means. Another method of obtaining herbicide-tolerant alleles is by selection in plant cell cultures.

Explants of plant tissue, e.g. embryos, leaf disks, etc. or actively growing callus or suspension cultures of a plant of interest are grown on medium in the presence of increasing concentrations of the inhibitory herbicide or an analogous inhibitor suitable for use in a laboratory environment. Varying degrees of growth are recorded in different cultures. In certain cultures, fast-growing variant colonies arise that continue to grow even in the presence of normally inhibitory concentrations of inhibitor. The frequency with which such faster-growing variants occur can be increased by treatment with a chemical or physical mutagen before exposing the tissues or cells to the inhibitor. Putative tolerance-conferring alleles encoding the ALS/AHAS protein are isolated and tested as described herein or using methods well known in the art. Those alleles identified as conferring herbicide tolerance may then be engineered for optimal expression and transformed into the plant. Alternatively, plants can be regenerated from the tissue or cell cultures containing these alleles.

A nucleotide substitution encoding an amino acid substitution of the present invention can also be introduced by directed mutagenesis techniques, such as homologous recombination and selected for based on the resulting herbicide-tolerance phenotype (see, e.g. Example 10, Paszkowski et al., EMBO J. 7. 4021-4026 (1988), and U.S. Pat. No. 5,487,992, particularly columns 18-19 and Example 8) or by oligonucleotide-based mutagenesis (see for example U.S. Pat. No. 5,563,350 or 5,756,325).

Accordingly, the present invention also discloses methods of obtaining a plant, plant cell or plant tissue tolerant to an inhibitor of ALS/AHAS activity. In a preferred embodiment, the method comprises introducing into said plant, plant cell or plant tissue a DNA molecule comprising a nucleotide sequence of the present invention, wherein said plant, plant cell or plant tissue is tolerant to said inhibitor. In another preferred embodiment, the method comprises introducing a nucleotide substitution of the present invention in a nucleotide sequence of said plant, plant cell or plant tissue which encodes a protein having ALS/AHAS activity, wherein the nucleotide substitution codes for an amino acid substitution conferring tolerance to an inhibitor of ALS/AHAS activity. Preferably, the nucleotide substitution is introduced in said nucleotide sequence by mutagenesis, preferably by chemical or physical mutagenesis, or by homologous recombination or oligonucleotide-based mutagenesis. The present invention thus also encompasses plants, plant cells or tissues comprising a nucleotide sequence encoding a protein having ALS/AHAS activity comprising an amino acid substitution of the present invention, wherein the plant, plant cell or tissue is obtained as described immediately above.

Transformation or mutagenesis of virtually any type of plant, both monocot and dicot is contemplated in the present invention. Among the crop plants for which transformation to herbicide tolerance is particularly contemplated are corn, wheat, rice, millet, oat, barley, sorghum, sunflower, sweet potato, alfalfa, sugar beet, Brassica species, tomato, pepper, soybean, tobacco, melon, squash, potato, peanut, pea, cotton, or cacao. The nucleotide sequences of the present invention may also be used to transform ornamental species, such as rose, and woody species, such as pine and poplar.

Various chemicals groups, such as the imidazolinones, sulfonylureas and triazolopyrimidines, are known as inhibitors of ALS/AHAS activity. In the present invention, preferred inhibitors of ALS/AHAS activity are imidazolinone herbicides (Hart et al. 1991, CRC Press. Pp. 247-256).

Examples of imidazolinone herbicides are imazapyr (Orwick et al., Proc. South. Weed Sci. Soc., Annu. Mtg., 36^{th}, 1983, p. 291), imazaquin (U.S. Pat. No. 4,798,619) and imazethapyr (U.S. Pat. No. 4,798,619). As described in the “Herbicide Handbook of the Weed Science Society of America”, 6th Ed., (1989), imazapyr (2-[4,5-dihydro-4-methyl-4-(1-methylethyl)-5-oxo-1H-imidazol-2-yl]-3-pyridinecarboxylic acid), is a non-specific, broad-spectrum herbicide, whereas both imazaquin (2-[4,5-dihydro-4-methyl-4-(1-methylethyl)-5-oxo-1H-imidazol-2-yl]-3-quinolinecarboxylic acid), and imazethapyr (2-[4,5-dihydro-4-methyl-4-(1-methylethyl)-5-oxo-1H-imidazol-2-yl]-5-ethyl-3-pyridinecarboxylic acid) are crop-specific herbicides. Other imidazolinone herbicides suitable for the present invention are for example Imazapic (Wixson et al. (1992), Proc. South. Weed Sci. Soc. 45, 341), Imazamox ((+)-5-methoxymethyl-2-(4-isopropyl-4-methyl-5-oxo-2-imidazolin-2-yl) nicotinic acid), Imazamethabenz-Me (U.S. Pat. No. 4,188,487).

Examples of sulfonylurea herbicides include 1-(2-chloro-phenylsulfonyl)-3-(4-methoxy-6-methyl-1,3,5-triazin-2-yl)urea (chlorsulfuron), 1-[(2-methoxycarbonylphenyl)-sulfonyl]-3-(4-methoxy-6-methyl-1,3,5-triazin yl-2-yl)urea (metsulfuron-methyl), 1-[(2-methoxycarbonyl-phenyl)sulfonyl]-3-methyl-3-(4-methoxy-6-methyl-1,3,5-triazin-2-yl)urea (DPX-L5300), 1-[(2-methoxycarbonylphenylmethyl)-sulfonyl]-3-(4,6-dimethoxypyrimid-2-yl) urea (bensulfuronmethyl), 1-[(2-methoxycarbonylphenyl)sulfonyl]-3-(di-methylpyrimid-2-yl)urea (sulfometuron-methyl), 1-[(2-methoxycarbonylthienyl)sulfonyl]-3-(4-methoxy-6-methyl-1,3,5-triazin-2-yl)urea (thiameturon-methyl), 1-[(2-ethoxycarbonylphenyl)sulfonyl]-3-(4-chloro-6-methoxypyrimid-2-yl)urea (chlorimuron-ethyl), 1-[(3-(N,N-dimethylamino-carbonyl)-pyrid-2-yl)sulfonyl]-3-(4,6-dimethoxypyrimid-2-yl)urea (nicosulfuron, SL 950), 1-[3-(ethylsulfonyl)-pyrid-2-yl)sulfonyl]-3-(4,6-dimethoxypyrimid-2-yl)urea (DPX-E9636), 1-[(2-(2-chloroethyl)-phenyl)sulfonyl]-3-(4-methoxy-6-methyl-1,3,5-triazin-2-yl)urea (triasulfuron), 1-[(2-methoxycarbonylphenyl)-sulfonyl]-3-(4,6-bis-(difluoromethoxy)pyrimid-2-yl)urea (pirimisulfuron), 1-[(4-ethoxycarbonyl-1-methyl-1,2-imidazol-5-yl)-sulfonyl]-3-(4,6-dimethoxypyrimid-2-yl)urea (pyrazosulfuron-methyl), 1-[(2-methoxycarbonylphenyl)sulfonyl]-3-(4-ethoxy-6-methylamino-1,3,5-triazin-2-yl)urea (DPX-A7881), cinosulfuron (CGA 142464), 1-[(3-trifluoroethylpyrid-2-yl)sulfonyl]-3-(4,6-dimethoxypyrimid-2-yl)urea (flazasulfuron, SL 160), 1-[(N-methyl-N-methylsulfonylamo)sulfonyl]-3-(4,6-dimethoxypyrimd-2-yl) urea (amidosulfuron, Hoe 75032),1-[(N-ethylsulfonyl-N-methylamino)sulfonyl]-3-(4,6-dimethoxypyrimid-2-yl)urea (SH-1),1-[(N-ethyl-N-ethylsulfonylamino)sulfonyl]-3-(4,6-dimethoxypyrimid-2-yl)urea (SH-2),1-[(2-ethoxy-phenoxy)sulfonyl]-3-(4,6-dimethoxypyrimid-2-yl)urea (SH-3),1-[(N-(dimethylaminosulfonyl)-N-methylamino)sulfonyl]-3-(4,6-dimethoxypyrimid-2-yl)urea (SH-4).

An amino acid substitution of the present invention preferably confers tolerance to an imidazolinone-type herbicide, as for example an imidazolinone herbicide listed above. The amino acid substitution may also confer cross-tolerance to other chemical groups of ALS/AHAS inhibitors, e.g. to sulfonylureas. Alternatively, an amino acid substitution of the present invention confers imidazolinone-specific tolerance, under which it is understood that the amino acid substitution does not confer substantial cross-tolerance to other chemical groups of ALS/AHAS inhibitors.

Plants according to the present invention, which are tolerant to an inhibitor of ALS/AHAS activity, are planted in a field and are used for the control of the growth of undesired vegetation in the field. Such methods of control comprise applying to a population of a plant disclosed above, or to the locus where such plants are grown, an effective amount of an inhibitor of ALS/AHAS activity. Preferably, the inhibitor of ALS/AHAS activity is an imidazolinone herbicide. Preferably, the plant is selected from the group consisting of sunflower, sugar cane, soybean, barley, cotton, tobacco, sugar beet, oilseed rape, maize, wheat, sorghum, rye, oats, turf and forage grasses, millet, forage and rice. In another preferred embodiment, the dose of inhibitor used is between 5 g and 1000 g active ingredient (a.i.) per hectare. Preferably, the dose is between 10 g and 200 g a.i. per hectare.

Herbicide tolerant plants or lines obtained using the present invention are particularly useful for the control of parasitic weed, e.g. broomrape, in sunflower, when an effective amount of an inhibitor of ALS/AHAS activity is applied thereto.

The present invention also discloses polymorphisms between herbicide-sensitive and herbicide-tolerant alleles. Using these polymorphisms, the inventors of the present invention have developed molecular markers used to breed tolerant plants, to follow the presence of the tolerance trait during breeding of plants or lines, and to control the presence of the tolerance trait in a commercial seeds lot. Such polymorphisms and markers are disclosed below. The polymorphisms described herein are preferably used in sunflower breeding, but may in some cases also used in other species.

In a preferred embodiment, a polymorphism of the present invention is within less than one centi-Morgan (cM) from the locus corresponding to the amino acid substitution conferring tolerance to an herbicide, more preferably within less than 0.1 cM, even more preferably within less than 0.05 cM, and thus tightly co-segregates with the amino acid substitution. Preferably, a polymorphism is located within the promoter region or termination region of a nucleotide sequence encoding an ALS/AHAS protein. Preferably, a polymorphism is located within the coding region of a nucleotide sequence encoding an ALS/AHAS protein. In a preferred embodiment, the inventors of the present invention disclose three consecutive silent nucleotide substitutions located at positions encoding amino acids 332 to 334 in SEQ ID NO:2 in a nucleotide sequence encoding a herbicide-tolerant ALS/AHAS. Particularly preferred polymorphism are nucleotide substitutions located at third-base positions in such codons, preferably at any one of positions 1229, 1232 or 1235 in SEQ NO: 1 (see e.g. example 5). Accordingly, the present invention discloses detections methods and kits based on such polymorphisms. In a preferred embodiment, the polymorphisms are used in sunflower.

In another preferred embodiment, a polymorphism disclosed by the inventors of the present invention is located within the codon encoding the amino acid substitution, which confers tolerance to herbicides. Such polymorphism comprises a nucleotide substitution at any position corresponding to positions 1566-1568 in SEQ ID NO:1. Particularly preferred polymorphisms are at positions corresponding to positions 1566-1567 in SEQ ID NO:1, more particularly at a position corresponding to position 1566 in SEQ ID NO:1. A particularly preferred polymorphism is a substitution of a “T” by a “C” at this position. A preferred marker disclosed in the present invention is a CAPS marker taking advantage of the creation of a new NspI restriction site by such “T” to “C” substitution. Accordingly, the present invention discloses detections methods and kits based on such polymorphisms, in particular based on an altered restriction digestion pattern (see for example example 4). Such polymorphism and molecular markers based thereupon, are used preferably in sunflower but may in somes cases also be used in other species.

In another preferred embodiment, the present invention discloses an isolated DNA molecule comprising about 15 successive nucleotides, preferably about 20 successive nucleotides, preferably about 50 successive nucleotides, of the nucleotide sequence set forth in SEQ ID NO:1 or SEQ ID NO:3, wherein said DNA molecule comprises a nucleotide polymorphism, wherein said polymorphism allows to differentiate between an allele conferring tolerance to an inhibitor of ALS/AHAS activity and an allele sensitive to said inhibitor. Such DNA molecules are preferably used as molecular markers. Preferably, the polymorphism is located at any one of positions 1229, 1232 or 1235 in SEQ NO: 1. Preferably, in this case, the DNA molecule comprises any one of the nucleotide sequences “GACTGTC” or “TACGGTT”, or a sequence complementary thereto. In another preferred embodiment, the polymorphism is located at any position corresponding to positions 1566-1568 in SEQ ID NO:1, more preferably at positions corresponding to positions 1566-1567 in SEQ ID NO:1, even more preferably at a position corresponding to position 1566 in SEQ ID NO:1. Preferably, in this case, the DNA molecule comprises any one of the nucleotide sequences “GCATGC” or “GTATGC”, or a sequence complementary thereto, or any one of the nucleotide sequences “CAGCATG”, “CAGTATG”, or a sequence complementary thereto.

Methods for detection of polymorphisms, which are well known in the art, are applied in the context of the present invention. Some of such methods or part thereof are described below. In particular, methods using the PCR amplification technique are contemplated within the scope of the present invention. A number of oligonucleotides are disclosed which are particularly useful in carrying out the present invention. For example, SEQ ID NO:8 (HiNK379) or SEQ ID NO:9 (HiNK415) is used to detect a polymorphism at the site of the amino acid substitution conferring tolerance to an herbicide. SEQ ID NO:10 (HiNK451), SEQ ID NO:11 (HiNK414), SEQ ID NO:12 (HiNK452) or SEQ ID NO:13 (HiNK415) are used to detect silent polymorphisms at positions corresponding to positions 1229, 1232 or 1235 in SEQ ID NO:1. Variations of the sequences of such nucleotides are however also contemplated and also form part of the instant invention. For example, addition or removal of a few nucleotides, for example 1-10 nucleotides (nt), preferably 1-5 nt, even more preferably 1-3 nt, at either end of the oligonucleotide are also contemplated. Also, a shift of the nucleotide on the nucleotide sequence of SEQ ID NO:1, for example by 1-10 nt, preferably 1-5 nt is also contemplated. Other preferred oligonucleotides of the present invention comprises about 6 successive nt from about position 1536 to about position 1596 in the nucleotide sequence set forth in SEQ ID NO:1 or SEQ ID NO:3, or in a sequence complementary thereto, or about 6 successive nt from about position 1199 to about position 1265 in the nucleotide sequence set forth in SEQ ID NO:1 or SEQ ID NO:3, or in a sequence complementary thereto.

Preferred oligonucleotides are about 8 to 50 nt long, more preferably 10 to 30 nt long, even more preferably 15 to 25 nt long.

DNA molecules comprising a site comprising a polymorphism of the present invention are preferably about 100-3,000 nt long, more preferably about 200 to 2,000 nt long, even more preferably about 500 to 1,500. Preferably, such DNA molecules are amplified PCR fragments.

In another preferred embodiment, the molecular markers of the present invention allow to differentiate between plants heterozygous or homozygous for the herbicide-tolerance allele.

Examples of such co-dominant markers are described in examples 4 and 5.

In another preferred embodiment, the present invention provides methods for mapping the tolerance trait in tolerant plants, for determining whether a herbicide-tolerant allele is present in a plant, and for transferring the tolerance to a susceptible or less tolerant plant. For example, the methods are used in breeding new plants or lines tolerant to herbicides or to control the quality of a seeds lot. The molecular markers disclosed herein allow for quicker release of herbicide tolerant lines to the market and for a better quality control of commercial seeds lots.

Spraying of plants with the herbicide is avoided or greatly reduced. Such methods are preferably used in sunflower breeding, but can also be applied for other crops.

In a preferred embodiment, a molecular marker of the present invention is beneficially used when herbicide-tolerant plants are selected from herbicide sensitive plants and is for example used on seeds originating from any self-pollination or cross-pollination of an herbicide tolerant plant. Herbicide-sensitive seeds originated from the cross and not carrying the herbicide tolerant allele or seeds contaminations accidentally present in a populations of seeds obtained from the cross are thus detected and can be set aside.

The molecular markers and methods of the present invention are also particularly beneficial in the various steps leading to a commercial variety, such as in breeding programs and in the seeds production process. For example, the present invention is used to introgress the herbicide tolerance allele and, optionally, to also introgress another trait that co-segregates with the herbicide tolerance trait, into a plant, for example an elite inbred line. The introgression of herbicide tolerance into the elite line is for example achieved by recurrent selection breeding, for example by backcrossing. In this case, the elite line (recurrent parent) is first crossed to a donor inbred (the non-recurrent parent) that carries the appropriate herbicide tolerance trait. The progeny of this cross is then mated back to the recurrent parent followed by selection in the resultant progeny for herbicide tolerance. After three, preferably four, more preferably five or more generations of backcrosses with the recurrent parent with selection for herbicide tolerance, the progeny is heterozygous for the locus harboring herbicide tolerance, but is like the recurrent parent for most or almost all other genes (see, for example, Poehlman & Sleper (1995) Breeding Field Crops, 4th Ed., 172-175; Fehr (1987) Principles of Cultivar Development, Vol. 1: Theory and Technique, 360-376, incorporated herein by reference). Selection for herbicide tolerance after each cross is carried out using the present invention, i.e. by testing seeds resulting from each cross with the molecular markers and methods of the present invention. In a preferred embodiment, the present invention therefore encompasses methods of breeding a herbicide tolerance allele into an elite line sensitive or less tolerant to the herbicide using the teachings of the present invention and comprising the following steps: crossing a first plant with a second plant, wherein one of the plants is tolerant to the herbicide, harvesting the seeds resulting from the cross, obtaining a sample of the seed or of a plant grown therefrom, detecting in said sample a DNA molecule of the present invention, the presence of said DNA molecule being indicative of an allele conferring tolerance to a herbicide, wherein said plant is tolerant to a herbicide and comprise the herbicide tolerant allele.

The present invention is also conveniently used in the production of stable homogenous inbred lines or cultivars (also sometimes called varieties), whereby a particular line is self-pollinated until satisfactory purity and homogeneity of the line is reached. The present invention is similarly used for the commercial production of seeds of a particular inbred line or cultivar.

Here again, after each cross a method of the present invention is applied to the seeds resulting from the cross and only herbicide tolerant plants are selected. In a preferred embodiment, the present invention therefore encompasses methods of producing herbicide tolerant seeds using the teachings of the present invention and comprising the following steps: crossing a herbicide tolerant plant with itself, harvesting the seeds resulting from the cross, obtaining a sample of the seed or of a plant grown therefrom, detecting in said sample a DNA molecule of the present invention, the presence of said DNA molecule being indicative of an allele conferring tolerance to a herbicide. In a preferred embodiment, the absence of a DNA molecule of the present invention in the sample, shows the absence of a herbicide-sensitive allele in said plant.

A co-dominant marker of the present invention is particularly useful for determining whether a plant to be screened is homozygous or heterozygous for the tolerant allele. Preferably, only homozygous plant are further used in the breeding or seeds production program. Such difference between homozygous or heterozygous plants cannot be detected by chemical sprays.

Current methods indeed require an additional cross to be able to differentiate between hetero- and homozygocity. The present invention therefore saves one cross which is of great advantage.

Similarly, the present invention is used in hybrid seed production. In this case, the present invention is used to assure that all hybrid seeds that germinate and grow in the field are herbicide tolerant. Preferably, molecular markers of the present invention also also used in quality assurance to ensure that the herbicide-tolerance trait is present in the hybrid seeds, and if desired, to ensure that the tolerance allele is in the homozygous state in hybrids seeds. In a preferred embodiment, the present invention therefore encompasses methods producing herbicide tolerance seeds using the teachings of the present invention and comprising the following steps: crossing a first plant with a second plant, wherein one of the plants is tolerant to the herbicide, harvesting the seeds resulting from the cross, obtaining a sample of the seed or of a plant grown therefrom, detecting in said sample a DNA molecule of the present invention, the presence of said DNA molecule being indicative of an allele conferring tolerance to a herbicide, wherein said sunflower plant is tolerant to a herbicide and comprise the herbicide tolerant allele. Co-dominant markers are preferably used.

The present invention therefore also disclose methods to determine whether a plant is homozygous or heterozygous for a herbicide-tolerant allele comprising obtaining a sample of a plant, detecting in said sample a DNA molecule of the present invention using a co-dominant marker, determining whether a nucleotide sequence encoding a herbicide tolerance ALS/AHAS of the present invention is heterozygous or homozygous in said plant.

The present invention thus provides a significant advancement to commercial breeding and seeds production processes using herbicide tolerance. Using the present invention large commercial quantities of herbicide tolerant seeds are produced with low impact on the environment and reduced cost.

Plant Transformation

A DNA molecule of the present invention is incorporated in plant or bacterial cells using conventional recombinant DNA technology. Preferably, this involves inserting the DNA molecule into an expression system to which the DNA molecule is heterologous (i.e., not normally present) using standard cloning procedures known in the art. The vector contains the necessary elements for the transcription and translation of the inserted protein-coding sequences in a host cell containing the vector. A large number of vector systems known in the art can be used, such as plasmids, bacteriophage viruses and other modified viruses. The components of the expression system may also be modified to increase expression. For example, truncated sequences, nucleotide substitutions, nucleotide optimization or other modifications may be employed. Expression systems known in the art can be used to transform virtually any crop plant cell under suitable conditions. A DNA molecule of the present invention is preferably stably transformed and integrated into the genome of the host cells. In another preferred embodiment, it is located on a self-replicating vector. Examples of self-replicating vectors are viruses, in particular gemini viruses. Transformed cells are regenerated into whole plants such that the DNA molecule confers herbicide tolerance in the transgenic plants.

A. Requirements for Construction of Plant Expression Cassettes

Gene sequences intended for expression in transgenic plants is first assembled in expression cassettes behind a suitable promoter expressible in plants. The expression cassettes may also comprise any further sequences required or selected for the expression of the heterologous DNA sequence. Such sequences include, but are not restricted to, transcription terminators, extraneous sequences to enhance expression such as introns and sequences intended for the targeting of the gene product to specific organelles and cell compartments. These expression cassettes can then be easily transferred to the plant transformation vectors described infra. The following is a description of various components of typical expression cassettes.

1. Promoters

The selection of the promoter used in expression cassettes will determine the spatial and temporal expression pattern of the heterologous DNA sequence in the plant transformed with this DNA sequence. Selected promoters will express heterologous DNA sequences in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the gene under various inducing conditions. Promoters vary in their strength, i.e., ability to promote transcription. Depending upon the host cell system utilized, any one of a number of suitable promoters known in the art can be used. For example, for constitutive expression, the CaMV 35S promoter, the rice actin promoter, or the ubiquitin promoter may be used. For regulatable expression, the chemically inducible PR-1 promoter from tobacco or Arabidopsis may be used (see, e.g., U.S. Pat. No. 5,689,044).

2. Transcriptional Terminators

A variety of transcriptional terminators are available for use in expression cassettes. These are responsible for the termination of transcription beyond the heterologous DNA sequence and its correct polyadenylation. Appropriate transcriptional terminators are those that are known to function in plants and include the CaMV ³⁵S terminator, the tml terminator, the nopaline synthase terminator and the pea rbcS E9 terminator. These can be used in both monocotyledonous and dicotyledonous plants.

3. Sequences for the Enhancement or Regulation of Expression

Numerous sequences have been found to enhance gene expression from within the transcriptional unit and these sequences can be used in conjunction with the genes of this invention to increase their expression in transgenic plants. For example, various intron sequences such as introns of the maize Adhl gene have been shown to enhance expression, particularly in monocotyledonous cells. In addition, a number of non-translated leader sequences derived from viruses are also known to enhance expression, and these are particularly effective in dicotyledonous cells.

4. Coding Sequence Optimization

The coding sequence of the selected gene may be genetically engineered by altering the coding sequence for optimal expression in the crop species of interest. Methods for modifying coding sequences to achieve optimal expression in a particular crop species are well known (see, e.g. Perlak et al., Proc. Natl. Acad. Sci. USA 88: 3324 (1991); and Koziel et al., Bio/technol. 11: 194 (1993)).

5. Targeting of the Gene Product within the Cell

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins which is cleaved during chloroplast import to yield the mature protein (e.g. Comai et al. J. Biol. Chem. 263: 15104-15109 (1988)). Other gene products are localized to other organelles such as the mitochondrion and the peroxisome (e.g. Unger et al. Plant Molec. Biol. 13: 411-418 (1989)).

The cDNAs encoding these products can also be manipulated to effect the targeting of heterologous products encoded by DNA sequences to these organelles. In addition, sequences have been characterized which cause the targeting of products encoded by DNA sequences to other cell compartments. Amino terminal sequences are responsible for targeting to the ER, the apoplast, and extracellular secretion from aleurone cells (Koehler & Ho, Plant Cell 2: 769-783 (1990)). Additionally, amino terminal sequences in conjunction with carboxy terminal sequences are responsible for vacuolar targeting of gene products (Shinshi et al. Plant Molec. Biol. 14: 357-368 (1990)). By the fusion of the appropriate targeting sequences described above to heterologous DNA sequences of interest it is possible to direct this product to any organelle or cell compartment.

B. Construction of Plant Transformation Vectors

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptII gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet 79: 625-631 (1990)), the hph gene, which confers resistance to the antibiotic hygromycin (Blochinger & Diggelmann, Mol Cell Biol 4: 2929-2931), the manA gene, which allows for positive selection in the presence of mannose (Miles and Guest (1984) Gene, 32:41-48; U.S. Pat. No. 5,767,378), and the dhfr gene, which confers resistance to methotrexate (Bourouis et al., EMBO J. 2(7): 1099-1104 (1983)), and the EPSPS gene, which confers resistance to glyphosate (U.S. Pat. Nos. 4,940,935 and 5,188,642).

1. Vectors Suitable for Agrobacterium Transformation

Many vectors are available for transformation using Agrobacterium tumefaciens. These typically carry at least one T-DNA border sequence and include vectors such as pBIN 19 (Bevan, Nucl. Acids Res. (1984)). Typical vectors suitable for Agrobacterium transformation include the binary vectors pCIB200 and pCIB2001, as well as the binary vector pCIB10 and hygromycin selection derivatives thereof. (See, for example, U.S. Pat. No. 5,639,949).

2. Vectors Suitable for Non-Agrobacterium Transformation

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. Typical vectors suitable for non-Agrobacterium transformation include pCIB3064, pSOG19, and pSOG35. (See, for example, U.S. Pat. No. 5,639,949).

C. Transformation Techniques

Once the coding sequence of interest has been cloned into an expression system, it is transformed into a plant cell. Methods for transformation and regeneration of plants are well known in the art. For example, Ti plasmid vectors have been utilized for the delivery of foreign DNA, as well as direct DNA uptake, liposomes, electroporation, micro-injection, and microprojectiles. In addition, bacteria from the genus Agrobacterium can be utilized to transform plant cells.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG- or electroporation-mediated uptake, particle bombardment-mediated delivery, or microinjection. In each case the transformed cells are regenerated to whole plants using standard techniques known in the art.

Transformation of most monocotyledon species has now also become routine. Preferred techniques include direct gene transfer into protoplasts using PEG or electroporation techniques, particle bombardment into callus tissue, as well as Agrobacterium-mediated transformation.

D. Plastid Transformation

In another preferred embodiment, a nucleotide sequence encoding a polypeptide having 1917, 2092, or 7724 activity is directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit expression levels that can readily exceed 10% of the total soluble plant protein. In a preferred embodiment, the nucleotide sequence is inserted into a plastid-targeting vector and transformed into the plastid genome of a desired plant host. Plants homoplasmic for plastid genomes containing the nucleotide sequence are obtained, and are preferentially capable of high expression of the nucleotide sequence.

Plastid transformation technology is for example extensively described in U.S. Pat. Nos. 5,451,513, 5,545,817, 5,545,818, and 5,877,462 in PCT application no. WO 95/16783 and WO 97/32977, and in McBride et al. (1994) Proc. Natl. Acad. Sci. USA 91, 7301-7305, all incorporated herein by reference in their entirety. The basic technique for plastid transformation involves introducing regions of cloned plastid DNA flanking a selectable marker together with the nucleotide sequence into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). The presence of cloning sites between these markers allowed creation of a plastid targeting vector for introduction of foreign genes (Staub, J. M., and Maliga, P. (1993) EMBO J. 12, 601-606).

Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab, Z., and Maliga, P. (1993) Proc. Natl. Acad. Sci. USA 90, 913-917).

Other selectable markers useful for plastid transformation are known in the art and encompassed within the scope of the invention.

Methods of Screening/Detecting Polymorphisms

Methods for screening for polymorphisms in nucleic acids for example include polymerase chain reaction (PCR), direct sequencing of nucleic acids, single strand polymorphism assay, ligase chain reaction, enzymatic cleavage, and southern hybridization.

Screening for nucleic acids can be accomplished by direct sequencing of nucleic acids. In fact, putative mutants identified by other methods may be sequenced to determine the exact nature of the mutation. Nucleic acid sequences can be determined through a number of different techniques which are well known to those skilled in the art. In order to sequence the nucleic acid, sufficient copies of the material must preferably be first amplified.

Amplification of a selected, or target, nucleic acid sequence may be carried out by any suitable means. (See generally Kwoh, D. and Kwoh, T., Am Biotechnol Lab, 8, 14 (1990)) Examples of suitable amplification techniques include, but are not limited to, polymerase chain reaction, ligase chain reaction (see Barany, Proc Natl Acad Sci USA 88, 189 (1991)), strand displacement amplification (see generally Walker, G. et al., Nucleic Acids Res. 20, 1691 (1992); Walker. G. et al., Proc Natl Acad Sci USA 89, 392 (1992)), transcription-based amplification (see Kwoh, D. et al., Proc Natl Acad Sci USA, 86, 1173 (1989)), self-sustained sequence replication (or “3SR”) (see Guatelli, J. et al., Proc Natl Acad Sci USA, 87, 1874 (1990)), the Q.beta. replicase system (see Lizardi, P. et al., Biotechnology, 6, 1197 (1988)), nucleic acid sequence-based amplification (or “NASBA”) (see Lewis, R., Genetic Engineering News, 12(9), 1 (1992)), the repair chain reaction (or “RCR”) (see Lewis, R., Genetic Engineering News, 12(9), 1 (1992)), and boomerang DNA amplification (or “BDA”) (see Lewis, R., Genetic Engineering News, 12(9), 1 (1992)). Polymerase chain reaction is currently preferred.

The present invention provides several methods for detecting Cleaved Amplified Polymorphic Sequences (CAPS; Konieczny et al., The Plant Journal 4(2):403-410, 1993) and for detecting Single Nucleotide Polymorphisms (SNPs) with a method termed “CAMPS” for Cleaved Amplified Modified Polymorphic Sequences, also known as dCAPS (Neff et al, 1998. Plant J. 14: 387-392; Michaels and Amasino, 1998. Plant J 14: 381-385). In the CAPS method, a nucleic acid containing a polymorphic restriction site is amplified using primers flanking the restriction site. The resulting PCR product is digested with the restriction endonuclease corresponding to the polymorphic restriction site, and the digested products are analyzed by gel electrophoresis.

In the CAMPS method, a nucleic acid molecule containing a single nucleotide polymorphism is mutagenized during PCR amplification to create a restriction endonuclease recognition site which includes the single nucleotide polymorphism. The resulting PCR product is digested with the corresponding restriction endonuclease, and the restriction endonuclease-treated products are analyzed for cleavage in a rapid high through-put assay.

The primers and oligonucleotides used in the methods of the present invention are preferably DNA, and can be synthesized using standard techniques and, when appropriate, detectably labeled using standard methods (Ausubel et al., supra). Detectable labels that can be used to tag the primers and oligonucleotides used in the methods of the invention include, but are not limited to, digoxigenin, fluorescent labels (e.g., fluorescein and rhodamine), enzymes (e.g., horseradish peroxidase and alkaline phosphatase), biotin (which can be detected by anti-biotin specific antibodies or enzyme-conjugated avidin derivatives), radioactive labels (e.g., .sup.32 P and sup. 125 I), calorimetric reagents, and chemiluminescent reagents. The labels used in the methods of the invention are detected using standard methods.

The specific binding pairs useful in the methods of the invention include, but are not limited to, avidin-biotin, streptavidin-biotin, hybridizing nucleic acid pairs, interacting protein pairs, antibody-antigen pairs, reagents containing chemically reactive groups (e.g., reactive amino groups), and nucleic acid sequence-nucleic acid binding protein pairs.

The solid supports useful in the methods of the invention include, but are not limited to, agarose, acrylamide, and polystyrene beads; polystyrene microtiter plates (for use in, e.g., ELISA); and nylon and nitrocellulose membranes (for use in, e.g., dot or slot blot assays).

Some methods of the invention employ solid supports containing arrays of nucleic acid probes. In these cases, solid supports made of materials such as glass (e.g., glass plates), silicon or silicon-glass (e.g., microchips), or gold (e.g., gold plates) can be used. Methods for attaching nucleic acid probes to precise regions on such solid surfaces, e.g., photolithographic methods, are well known in the art, and can be used to make solid supports for use in the invention. (For example, see, Schena et al., Science 270:467-470, 1995; Kozal et al., Nature Medicine 2(7):753-759, 1996; Cheng et al., Nucleic Acids Research 24(2):380-385, 1996; Lipshutz et al., BioTechniques 19(3):442-447, 1995; Pease et al., Proc. Natl. Acad. Sci. USA 91:5022-5026, 1994; Fodor et al., Nature 364:555-556, 1993; Pirrung et al., U.S. Pat. No. 5,143,854; and Fodor et al., WO 92/10092.)

In general, DNA amplification techniques such as the foregoing involve the use of a probe, a pair of probes, or two pairs of probes which specifically bind to DNA encoding the gene of interest, but do not bind to DNA which does not encode the gene, under the same hybridization conditions, and which serve as the primer or primers for the amplification of the gene of interest or a portion thereof in the amplification reaction.

Nucleic acid sequencing can be performed by chemical or enzymatic methods. The enzymatic method relies on the ability of DNA polymerase to extend a primer, hybridized to the template to be sequenced, until a chain-terminating nucleotide is incorporated. The most common methods utilize didoexynucleotides. Primers may be labelled with radioactive or fluorescent labels. Various DNA polymerases are available including Klenow fragment, AMV reverse transcriptase, Thermus aquaticus DNA polymerase, and modified T7 polymerase.

Recently, single strand polymorphism assay (“SSPA”) analysis and the closely related heteroduplex analysis methods have come into use as effective methods for screening for single-base polymorphisms (Orita, M. et al., Proc Natl Acad Sci USA, 86, 2766 (1989)). In these methods, the mobility of PCR-amplified test DNA from test sources is compared with the mobility of DNA amplified from control sources by direct electrophoresis of samples in adjacent lanes of native polyacrylamide or other types of matrix gels. Single-base changes often alter the secondary structure of the molecule sufficiently to cause slight mobility differences between the normal and mutant PCR products after prolonged electrophoresis.

Ligase chain reaction is yet another recently developed method of screening for mutated nucleic acids. Ligase chain reaction (LCR) is also carried out in accordance with known techniques. LCR is especially useful to amplify, and thereby detect, single nucleotide differences between two DNA samples. In general, the reaction is called out with two pairs of oligonucleotide probes: one pair binds to one strand of the sequence to be detected; the other pair binds to the other strand of the sequence to be detected. The reaction is carried out by, first, denaturing (e.g., separating) the strands of the sequence to be detected, then reacting the strands with the two pairs of oligonucleotide probes in the presence of a heat stable ligase so that each pair of oligonucleotide probes hybridize to target DNA and, if there is perfect complementarity at their junction, adjacent probes are ligated together. The hybridized molecules are then separated under denaturation conditions. The process is cyclically repeated until the sequence has been amplified to the desired degree. Detection may then be carried out in a manner like that described above with respect to PCR.

Southern hybridization is also an effective method of identifying differences in sequences. Hybridization conditions, such as salt concentration and temperature can be adjusted for the sequence to be screened. Southern blotting and hybridizations protocols are described in Current Protocols in Molecular Biology (Greene Publishing Associates and Wiley-Interscience), pages 2.9.1-2.9.10. Probes can be labelled for hybridization with random oligomers (primarily 9-mers) and the Klenow fragment of DNA polymerase. Very high specific activity probe can be obtained using commercially available kits such as the Ready-To-Go DNA Labelling Beads (Pharmacia Biotech), following the manufacturer's protocol. Briefly, 25 ng of DNA (probe) is labelled with .sup.32 P-dCTP in a 15 minute incubation at 37.degree. C. Labelled probe is then purified over a ChromaSpin (Clontech) nucleic acid purification column.

Possible competition of probes having high repeat sequence content, and stringency of hybridization and washdown will be determined individually for each probe used. Alternatively, fragments of a candidate gene may be generated by PCR, the specificity may be verified using a rodent-human somatic cell hybrid panel, and subcloning the fragment. This allows for a large prep for sequencing and use as a probe. Once a given gene fragment has been characterized, small probe preps can be done by gel- or column-purifying the PCR product.

These mismatch detection protocols use samples generated by PCR and thus require use of very little genomic template. All of these methods can provide very good clues regarding the location of the sequence change which leads to the appearance of anomalous bands, hence facilitating subsequent cloning and sequencing strategies.

Methods used in the present invention to detect polymorphisms also include Taqman (Livak, 1999, Genet Anal 14: 143-149), FRET (Chen et al, 1998, Genome Res 8:549-546) and Pyrosequencing (Ahmadian et al, 2000, Anal Biochem 280: 103-110; Alderborn et al, 2000, Genome Res 10: 1249-1258; Nordstrom et al., 2000, Biotechnol Appl Biochem 31: 107-112).

Methods of screening for mutated nucleic acids can be carried out using either deoxyribonucleic acids (“DNA”) or messenger ribonucleic acids (“mRNA”) isolated from the biological sample. During periods when the gene is expressed, mRNA may be abundant and more readily detected. However, these genes are temporally controlled and, at most stages of development, the preferred material for screening is DNA.

Alternatively, the detection of a mutated gene is carried out by collecting a biological sample and testing for the presence or form of the protein produced by the gene. The mutation in the gene may result in the production of a mutated form of the peptide or the lack of production of the gene product. In this embodiment, the determination of the presence of the polymorphic form of the protein can be carried out, for example, by isoelectric focusing, protein sizing, or immunoassay. In an immunoassay, an antibody that selectively binds to the mutated protein can be utilized. Such methods for isoelectric focusing and immunoassay are well known in the art, and are discussed in further detail below.

Changes in the size or charge of the polypeptide can be identified by isoelectric focusing or protein sizing techniques. Changes resulting in amino acid substitutions, where the substituted amino acid has a different charge than the original amino acid, can be detected by isoelectric focusing. Isoelectric focusing of the polypeptide through a gel having an ampholine gradient at high voltages separates proteins by their pI. The pH gradient gel can be compared to a simultaneously run gel containing the wild-type protein. Protein sizing techniques such as protein electrophoresis and sizing chromatography can also be used to detect changes in the size of the product.

As an alternative to isoelectric focusing or protein sizing, the step of determining the presence of the mutated polypeptides in a sample may be carried out by an antibody assay with an antibody which selectively binds to the mutated polypeptides (i.e., an antibody which binds to the mutated polypeptides but exhibits essentially no binding to the wild-type polypeptide without the polymorphism in the same binding conditions).

Antibodies used to bind selectively the products of the mutated genes can be produced by any suitable technique. For example, monoclonal antibodies may be produced in a hybridoma cell line according to the techniques of Kohler and Milstein, Nature, 265, 495 (1975), which is hereby incorporated by reference. A hybridoma is an immortalized cell line which is capable of secreting a specific monoclonal antibody. The mutated products of genes which are associated with autism may be obtained from a human patient, purified, and used as the immunogen for the production of monoclonal or polyclonal antibodies. Purified polypeptides may be produced by recombinant means to express a biologically active isoform, or even an immunogenic fragment thereof may be used as an immunogen. Monoclonal Fab fragments may be produced in Escherichia coli from the known sequences by recombinant techniques known to those skilled in the art. (See, e.g., Huse, W., Science 246, 1275 (1989)) (recombinant Fab techniques).

The invention will be further described by reference to the following detailed examples. These examples are provided for purposes of illustration only, and are not intended to be limiting unless otherwise specified.

### EXAMPLES

### Example 1

### Introgression of Imidazolinone Tolerance into Cultivated Sunflower (H. annuus)

A imidazolinone tolerant sunflower accession (released by the USDA (Al-Khatib et al, 1998, Weed Sci 46: 403-407) is introgressed into an elite parental line by two cycles of backcrossing followed by two selfings. Tolerant progeny plants are selected by herbicide treatments using a single application of 140-280 g/ha of imazamox (brand name Raptor commercialized by American Cyanamid) at the 4-5 leaf stage. Herbicide treatment of the final BC2S2 progenies allows the identification of homozygous tolerant family, which are used for the subsequent amplification and cloning of a nucleotide sequence encoding a sunflower ALS/AHAS (HaALS).

### Example 2

### Amplification and Sequencing of a Nucleotide Sequence Encoding a Sunflower ALS/AHAS

ClustalW alignment (Thompson et al, 1994, Nucl Acids Res 22: 4673-46780) of nucleotide sequences encoding a sunflower ALS/AHAS for Amaranthus sp. (GenBank Accession Number U55852), Arabidopsis thaliana (X51514), Bassia scoparia (AF094326), Brassica napus (X116708), Gossypium hirsutum and Nicotiana tabacum (X07645), allows the identification of conserved sequence motifs (Table 1) and the design of degenerate primers against those motifs. Using such degenerate primers:

| | |
|---|---|
| HiNK366: | |
| 5′ GTNTTYGCBTACCCWGGHGG 3′, | (SEQ ID NO: 5) |
| HiNK369: | |
| 5′ GCCCACATYTGRTGYTGCCCRACACC 3′, | (SEQ ID NO: 6) |
| HiNK370: | |
| 5′ ACATGYTCYTGRTGHGGDMMRATCACATC 3′, | (SEQ ID NO: 7) |

the central region of the HaALS gene spanning 1.6 Kb is amplified, cloned and sequenced in two consecutive steps. First, primers HiNK366 and HiNK369 allow the successful amplification of a fragment of 1.15 Kb. Cloning and sequencing of this fragment allows the design of the non-degenerate primer HiNK379 (5′ GAGGCTTTTTATCTTGCGAGTTC 3′, SEQ ID NO:8) that is combined with degenerate primer HiNK370 to yield a second, but partially overlapping ALS/AHAS fragment of 1.23 Kb. Together both fragments comprise 1.60 Kb). The gene sequence is completed by means of TAIL-PCR (Liu and Whittier, 1995, Genomics 25: 674-681; Liu et al, 1995, Plant J 8: 457-463), yielding the 5′ and 3′ extremities of the coding region, as well as 0.23 Kb respectively 0.75 Kb of flanking sequences. The nucleotide and amino acid sequences of the herbicide tolerant ALS/AHAS and flanking sequences are shown in SEQ ID NO:3 and SEQ ID NO:4, respectively.

### Example 3

### Identification of the Mutation Underlying Herbicide Tolerance in Sunflower

Surprisingly, the amino acid sequence of the tolerant ALS/AHAS does not show any of the mutations known in the literature to confer tolerance to imidazolinone and/or chlorsulfuron in other crop species (Wright et al, 1998, Weed Sci 46: 13-23). The amplification and sequence analysis of the ALS/AHAS protein from five susceptible inbred lines and their subsequent alignment to the tolerant ALS/AHAS protein results in the final identification of the mutation underlying the herbicide tolerance in sunflower (Table 2): Y445H resulting from the single nucleotide substitution T 1332C. Amongst the six ALS/AHAS protein sequences studied, Y445H is the only amino acid substitution correlating with the observed herbicide tolerance; all others represent genetic variability at the ALS/AHAS without affecting herbicide tolerance.

### Example 4

### Development of Co-Dominant Caps Marker for Herbicide Tolerance

The nucleotide substitution causing the Y445H mutation reveals to create a Nsp I restriction site that is exploited to create a co-dominant CAPS marker for imidazolinone tolerance in sunflower. Amplification of the coding region comprising the Y445H substitution and the polymorphic Nsp I restriction site using primers

| | |
|---|---|
| HiNK379: | |
| 5′ GAGGCTTTTTATCTTGCGAGTTC 3′ | (SEQ ID NO: 8) |
| and | |
| HiNK415: | |
| 5′ CCTTAGAGAACATTATCACTCGC 3′, | (SEQ ID NO: 9) |

followed by Nsp I digestion of the obtained amplification products, yields the expected polymorphism regarding the size of the restriction fragments when comparing a number of susceptible sunflower lines to the tolerant accession. Besides the Nsp I restriction site resulting from the Y445H mutation, the amplification product of the tolerant HaALS allele obtained with primers HiNK379 and HiNK415 comprises one additional Nsp I restriction site at 174 nucleotides upstream of the diagnostic Nsp I site, but that is not correlated to the imidazolinone tolerance. The predicted size of the Nsp I restriction fragments for the tolerant allele are calculated at 0.17 Kb, 0.51 Kb and 0.83 Kb; those for the susceptible allele at 1.5 Kb or at 0.51 Kb and 1.0 Kb depending on the presence of the non-diagnostic Nsp I site.

Application of this CAPS marker in backcross breeding programs for imidazolinone tolerance proves successful. Backcross populations typically comprise homozygous susceptible and heterozygous tolerant individuals in equal frequencies. Genotyping of five susceptible and five tolerant plants from a said backcross population for imidazolinone tolerance yields the expected restriction profiles. The susceptible plants all show one single band at 1.5 Kb corresponding to the susceptible allele. The tolerant plants showed four bands, the lower three bands resulting from the restriction of the amplification product from the tolerant allele that was digested twice, the upper band at 1.5 Kb corresponding to the non-digested susceptible allele.

### Example 5

### Development of a Co-Dominant PCR-Marker by Means of Post-PCR Multiplexing

Alignment of the tolerant HaALS allele to the available sequences of susceptible alleles shows the Y445H mutation to be linked to three consecutive silent or third-base mutations at amino acid positions 332 to 334. At the nucleotide sequence level, a thyrine at position 1229 in the susceptible allele is replaced by a guanine in the tolerance allele, a guanine at position 1232 in the susceptible allele is replaced by a thymine in the tolerance allele, a thymine at position 1235 in the susceptible allele is replaced by a cytosine in the tolerance allele. This polymorphic sequence motif is exploited to design two allele-specific primers:

| | | |
|---|---|---|
| HiNK451: | 5′ GGATGCATGGGACTGTC 3′, | (SEQ ID NO: 10) |
| and | | |
| HiNK452: | 5′ GGATGCATGGTACGGTT 3′, | (SEQ ID NO: 11) |

for the tolerant and susceptible HaALS allele respectively. When combined with an appropriate primer, these primers allow the allele-specific amplification of the HaALS gene, but cannot distinguish homozygous from heterozygous plants. In order to render the assay co-dominant, the allele-specific primers are combined with different primers hybridizing at different positions downstream of the stop codon:

| | |
|---|---|
| HiNK414: | |
| 5′ CCGAAACTTTGACCCGTTACC 3′ | (SEQ ID NO: 12) |
| HiNK415: | |
| 5′ CCTTAGAGAACATTATCACTCGC 3′ | (SEQ ID NO: 13) |

The combination of primers HiNK451 and HiNK414 yields an amplification product of 1.5 Kb specific for the tolerant allele, primers HiNK452 and HiNK415 a product of 1.2 Kb specific for the susceptible allele. Consequently, the co-dominant marker consists of two separate PCR reactions yielding amplification products of different sizes that may be combined or multiplexed before gel electrophoresis. Application of this marker proves successful when genotyping backcross populations or selfings for imidazolinone tolerance in sunflower, allowing the distinction of homozygous susceptible, homozygous tolerant and heterozygous tolerant individuals.

### Example 6

### Development of a 5′ Nuclease Assay for Imidazolinone Resistance

The CAPS marker described in Example 4 was converted into a 5′ nuclease assay (Lie et al, 1998, Curr Opinion in Biotechnol 9: 43-48) by designing Taqman MGB probes targeting the Single Nucleotide Polymorphism (SNP) underlying the Y445H mutation (the nucleotides constituting the SNP are underlined):

| | |
|---|---|
| HiNK702: | |
| VIC 5′ | |
| CTTGAATAGCATACTGTGGA 3′ MGB-NFQ | (SEQ ID NO: 14) |
| and | |
| HiNK703: | |
| FAM 5′ | |
| TTGAATAGCATGCTGTGGA 3′ MGB-NFQ | (SEQ ID NO: 15) |

Probe HiNK702 hybridizes to the susceptible allele and carries the VIC reporter dye, HiNK703 hybridizes to the resistant allele and carries the FAM reporter dye. At their 3′ end both probes contain the Minor Groove Binding (MGB) protein (Kutyavin et al, 2000, Nucl Acids Res 28: 655-661), as well as the Non Fluorescent Quencher (NFQ) molecule. The Taqman MGB probes are used in combination with primers

| | | |
|---|---|---|
| HiNK700: | 5′ CATTCCCGCCCGTTAACTC 3′ | (SEQ ID NO: 16) |
| and | | |
| HiNK701: | 5′ GTTTGGCGAAGCGATTCCT 3′, | (SEQ ID NO: 17) |

that specifically amplify the region of the ALS gene carrying the Y445H mutation. Probes and primers were designed using the Primer Express 2.0 software distributed by Applied Biosystems Inc. The assay is run in a total reaction volume of 10 μl containing 1.65 mM MgCl₂, 0.25 mM dNTPs, 100 nM of both Taqman MGB probes, 200 nM of both primers, 150 nM of sulforhodamine (ROX as passive reference) and 0.2 U of Taq Platinum polymerase from Invitrogen Inc. The amplification program starts off with a hot start of 10 minutes at 95° C., followed by 40 cycles of 15 seconds at 95° C. and 1 minute at 60° C.

After the PCR reaction, the amplifications are scanned at a PRISM 7900 from Applied Biosystems Inc. for both FAM and VIC fluorescence at 520 nm and 550 nm respectively. FAM fluorescence is associated with the resistant allele, VIC fluorescence is associated with the susceptible allele.

The 5′ nuclease assay is used for allele discrimination of the ALS gene in sunflower breeding programs for imidazolinone resistance. Using the SDS 2.0 software from Applied Biosystems Inc, the samples are grouped into three separate classes corresponding to the homozygous susceptible (rr), homozygous resistant (RR) and heterozygous resistant (Rr) individual plants.

### Example 7

### Transgenic Expression of a Nucleotide Sequence Encoding a Herbicide Tolerant ALS/AHAS

The nucleotide sequences encoding a tolerant and a susceptible sunflower ALS/AHAS are assembled into plant expression cassettes and transformed to Arabidopsis. The promoter and terminator driving the ALS/AHAS coding sequences comprise the Ubi3 promoter from Arabidopsis (Norris et al, 1993, Plant Mol Biol 21: 895-906) and the nos terminator from Agrobacterium tumefaciens respectively. Each expression cassette is transferred to the T-DNA of a binary vector carrying the PAT selectable marker gene under the control of the CaMV 35S promoter and terminator, and the resulting transformation vectors transferred to Agrobacterium tumefaciens strain EHA101 (Hood et al, 1986. J Bacteriology 168: 1291-1301). Flowering Arabidopsis plants are transformed using the floral dip method for in vivo transformation (Clough and Bent, 1998, Plant J 16: 735-743), seeds harvested, and transgenic plants selected by Basta treatment of the progeny plants. The herbicide tolerance of the transformed plants is determined.

TABLE 1

Clustal W(1.4) multiple sequence alignment
Clustal W alignment of the nucleotide sequences encoding
ALS/AHAS for six dicotyledonous plants species: Amaranthus sp.
(U55852), Arabidopsis thaliana (X51514), Bassia scoparia
(AF094326), Brassica napus (X16708), Gossypium hirsutum and
Nicotiana tabacum (X07645), including the deduced consensus
sequence (bottom line). The sequence and position of the de-
generate primers used to amplify the ALS/AHAS gene from sun-
flower are shown in bold and underlined: HiNK366 (nucleotides
347-366), HiNK369 (nucleotides 1469-1494, complementary sense)
and HiNK370 (nucleotides 1931-19580, complementary sense).
Pairwise Alignment Parameters:
ktup = 2 Gap Penalty = 5 Top Diagonals = 4 Window Size = 4
Multiple Alignment Parameters:
Open Gap Penalty = 10.0 Extend Gap Penalty = 5.0
Delay Divergent = 40% Transitions: Weighted
Alignment Score = 152705; Conserved Identities = 930; Gaps
Inserted = 29

| | |
|---|---|
| | 10 20 30 40 50 |
| Amaranthus | ATGGTCACTCTCAACCACATTTCCTCTTT |
| Arabidopsis | ATGGCGGCGGCAACAACAACAACAACAACATCTTCTTCGATCTCCTTCTC |
| Bassia | ATGGCGTCTACTTGTGCAAATCCCACTTT |
| Brassica | |
| Gossypium | ATGGCGGCTGCCACTGCGAACTCCGCTTTGCCTAA |
| Nicotiana | ATGGCGGCGGCTGCGGCGGCTCCATCTCCCTCTTTCTC |
| | 60 70 80 90 100 |
| Amaranthus | TACTAAACCTAACAAAACGTATCTGCAATCATCCATTTACGCTATC---- |
| Arabidopsis | CACCAAACCATCTCCTTCCTCCTCCAAATCACCATTACCAATCTCCAGAT |
| Bassia | TACCCCTTTCACCAGTAAACCCCTTAAACC--CCGTTCTCCCTTTC---- |
| Brassica | ATGGCTTCGTTTTCGTTCTTCGGCACCATTCCGTCGTCTCCCACAAAAG |
| Gossypium | GCTTTCTACGTTAACTTCCTCCTTCAAATCTTCCATACCCATTTCCAAAT |
| Nicotiana | CAAAACCCTATCGTCCTCCTCCTCCAAATCCTCCACCCTCCTCCCTAGAT |
| | C |
| | 110 120 130 140 150 |
| Amaranthus | CCTTTTTCCAATTCTCTTAAACCCACTTCTTCTTCTTCAATCCTCGCCGC |
| Arabidopsis | TCTCCCTCCCATTCTCCCTAAACCCCAACAAATCATCCTCCTCCTCCCGC |
| Bassia | ACTCTTTCCCATTTCCCTCAAACCCCAAAACCCCTTCCTCTTCATTTCGC |
| Brassica | CTTCCGTCTTCTCCCTGCCGGTGTCGGTAACTACGCTCCCGTCCTTCCCG |
| Gossypium | CCAGCCTTCCCTTCTCCACAACCCCTCAAAA---GCCCACCCCTTACCGC |
| Nicotiana | CCACCTTCCCTTTCCCCCACCACCCCCACAAAACCACCCCACCACCCCTC |
| | T T C C C |
| | 160 170 180 190 200 |
| Amaranthus | CCTCTTCAAATCTCATCATCTTCTTCTACAATCACCTAAACCTAAACCTC |
| Arabidopsis | CGCCGCGGTATCAAATCCAGCTCTCCCTCCTCCATCTCCGCCGTGCTCAA |
| Bassia | AACCTCAAAATCACATCTTCTCTCTCTTCT-TCACAACCCCCGAAACCAC |
| Brassica | CGCCGCCGTGCTA---------CTCGTGTCTCCGTTTCCGCCA-ACTCGA |
| Gossypium | TCCTTCGACGTTT---------CCTGCTCTCTTTCTCATGCAAGCTCTAA |
| Nicotiana | CACCTCACCCCCAC--------CCACATTCACAGCCAACGCCGTCGTTTC |
| | C |
| | 210 220 230 240 250 |
| Amaranthus | CTTCCGCTACTATAACTCA---ATCACCTTCGTCTCTCACCGATGATAAA |
| Arabidopsis | CACAACCACCAATGTCACAA----CCACTCCCTCTCCAACCAAACCTACC |
| Bassia | CTTCCGCCGTCAAAACCCACTCACCACCTTCCCCTCTCACAACCGACGAA |
| Brassica | -AGAAAGACCAAGACCGCA-----CAGCTTCACGTCGAGAGAATCCGAGC |
| Gossypium | CCCCAGATCCGCCGCCACAT----CCGTTACCCCAAAAAATGCTCCTCCC |
| Nicotiana | -ACCATCTCCAATGTCATTTCCACTACCCAAAAAGTTTCCGAGACCCAAA |
| | C |
| | 260 270 280 290 300 |
| Amaranthus | CCCTCTTC---TTTTGTTTCCCGATTTAGCCCTGAAGAACCCAGAAAAGG |
| Arabidopsis | AAACCCGAAACATTCATCTCCCGATTCGCTCCAGATCAACCCCGCAAAGG |
| Bassia | CCCCCGCAAGGTTTTGTTTCCCGATTTGCCCCTGACCAACCCAGAAAAGG |
| Brassica | A---------CATTCAGCTCCAAATACGCTCCCAACGTGCCCCGCAGTGG |
| Gossypium | CAT------GATTTCATTTCCAGGTATGCGGATGATGAGCCCCGGAAAGG |
| Nicotiana | AAGCC-GAAACTTTCGTTTCCCGTTTTGCCCCTGACGAACCCAGAAAGGG |
| | TT TCC T A CCC G A GG |
| | 310 320 330 340 350 |
| Amaranthus | TTGCGATGTTCTCGTTGAAGCTCTTGAACGTGAAGGTGTTACCGATGTTT |
| Arabidopsis | CGCTGATATCCTCGTCGAAGCTTTAGAACGTCAAGGCGTAGAAACCGTAT |
| Bassia | CTGCGATGTCCTCGTTGAGGCCCTCGAGCGGGAGGGCGTCACCGACGTGT |
| Brassica | CGCAGACATCCTGGTCGAAGCCCTGGAGCGTCAAGGAGTGGACGTAGTCT |
| Gossypium | CGCTGATATCCTGGTGGAAGCCCTGGTTCGTGAAGGGGTCAAGGATGTGT |
| Nicotiana | TTCCGACGTTCTCGTGGAGGCCCTCGAAAGAGAAGGGGTTACGGACGTTT |
| | GA T CT GT GA GC T G G A GG GT GTNT |
| | 360 370 380 390 400 |
| Amaranthus | TTGCTTACCCTGGTGGAGCTTCCATGGAAATCCATCAAGCTCTTACTCGT |
| Arabidopsis | TCGCTTACCCTGGAGGTGCATCAATGGAGATTCACCAAGCCTTAACCCGC |
| Bassia | TCGCTTATCCTGGTGGCGCATCAATGGAGATTCATCAAGCTCTGACTCGC |
| Brassica | TCGCTTACCCAGGAGGCGCATCAATGGAGATCCATCAAGCCCTAACTCGC |
| Gossypium | TTGCCTACCCAGGTGGAGCTTCAATGGAGATCCACCAGGCTTTAACCCGC |
| Nicotiana | TTGCGTACCCAGGCGGCGCTTCCATGGAGATTCACCAAGCTTTGACGCGC |
| | TYGCBTACCCWGGHGG GC TC ATGGA AT CA CA GC T AC CG |
| | 410 420 430 440 450 |
| Amaranthus | TCTAATATCATTAGAAATGTTCTTCCTCGACATGAACAAGGTGGGGTTTT |
| Arabidopsis | TCTTCCTCAATCCGTAACGTCCTTCCTCGTCACGAACAAGGAGGTGTATT |
| Bassia | TCTGATTCCATACGCAACGTCCTGCCTCGCCACGAGCAAGGCGGGATCTT |
| Brassica | TCCAACACAATCCGAAACGTCCTTCCCCGTCACGAACAAGGAGGTATCTT |
| Gossypium | TCAAAAATCATCCGAAATGTCCTTCCGCGACACGAGCAAGGTGGGGTCTT |
| Nicotiana | TCAAGCATCATCCGCAACGTGCTACCACGTCACGAGCAGGGTGGTGTCTT |
| | TC AT G AA GT CT CC CG CA GA CA GG GG T TT |
| | 460 470 480 490 500 |
| Amaranthus | CGCTGCTCAAGGCTACGCTCGTGCTACTGGACGTGTTGGAGTTTGTATTG |
| Arabidopsis | CGCAGCAGAAGGATACGCTCGATCCTCAGGTAAACCAGGTATCTGTATAG |
| Bassia | TGCCGCGGAGGGGTATGCTCGTGCCACGGGCCGTGTTGGTGTCTGCATTG |
| Brassica | CGCCGCCGAGGGTTACGCTCGTTCCTCCGGTAAACCCGGAATCTGCATCG |
| Gossypium | TGCCGCCGAGGGCTACGCGCGCTCCTCTGGCATTCCCGGCGTTTGCATTG |
| Nicotiana | CGCCGCTGAGGGTTACGCACGCGCCACCGGCTTCCCCGGCGTTTGCATTG |
| | GC GC A GG TA GC CG C C GG GG T TG AT G |
| | 510 520 530 540 550 |
| Amaranthus | CCACTTCTGGTCCGGGTGCTACTAATCTTGTTTCCGGTTTTGCTGATGCA |
| Arabidopsis | CCACTTCAGGTCCCGGAGCTACAAATCTCGTTAGCGGATTAGCCGATGCG |
| Bassia | CGACATCTGGCCCTGGCGCTACGAACCTCGTGTCCGGGTTTGCTGATGCT |
| Brassica | CCACTTCCGGTCCAGGAGCTATGAATCTCGTCAGCGGATTAGCCGACGCC |
| Gossypium | CGACGTCTGGCCCTGGGGCAACCAACTTGGTGAGTGGTCTCGCTGATGCA |
| Nicotiana | CCACCTCCGGCCCTGGCGCCACCAATCTCGTCAGTGGCCTCGCGGACGCC |
| | C AC TC GG CC GG GC A AA T GT GG T GC GA GC |
| | 560 570 580 590 600 |
| Amaranthus | CTTCTTGACTCAGTCCCGCTTGTCGCCATTACTGGGCAAGTTCCTCGGCG |
| Arabidopsis | TTGTTAGATAGTGTTCCTCTTGTAGCAATCACAGGACAAGTCCCTCGTCG |
| Bassia | TTGCTCGATTCCGTTCCACTGGTGGCGATCACGGGGCAGGTGCCGCGGCG |
| Brassica | CTGTTTGACAGCGTACCCCTCATCGCAATCACAGGACAGGTCCCTCGCCG |
| Gossypium | ATGCTCGATAGTATCCCTCTCGTGGCGATCACTGGTCAAGTCCCTCGTCG |
| Nicotiana | CTACTGGATAGCGTCCCCATTGTTGCTATAACCGGTCAAGTGCCACGTAG |
| | T T GA T CC T T GC AT AC GG CA GT CC CG G |
| | 610 620 630 640 650 |
| Amaranthus | TATGATTGGTACTGATGCTTTTCAAGAGACACCTATAGTTGAGGTAACAC |
| Arabidopsis | TATGATTGGTACAGATGCGTTTCAAGAGACTCCGATTGTTGAGGTAACGC |
| Bassia | AATGATTGGGACGGATGCTTTTCAGGAGACTCCTATTGTTGAGGTAACAC |
| Brassica | GATGATTGGTACCATGGCGTTCCAGGAGACACCCGTTGTTGAGGTAACGA |
| Gossypium | GATGATCGGTACCGATGCTTTCCAGGAAACTCCAATTGTTGAGGTAACAA |
| Nicotiana | GATGATCGGTACTGATGCTTTTCAGGAAACTCCGATTGTTGAGGTAACTA |
| | ATGAT GG AC GC TT CA GA AC CC T GTTGAGGTAAC |
| | 660 670 680 690 700 |
| Amaranthus | GATCAATTACTAAGCATAATTATTTGGTGTTAGATGTTGAGGATATCCCT |
| Arabidopsis | GTTCGATTACGAAGCATAACTATCTTGTGATGGATGTTGAAGATATCCCT |
| Bassia | GGTCTATTACCAAGCATAATTATCTGGTATTAGATGTTGAGGATATTCCT |
| Brassica | GGACTATAACGAAACATAACTATCTTGTTATGGAAGTTGATGATATACCT |
| Gossypium | GGTCTATTACGAAGCATAATTATCTTGTTCTTGATGTGGATGATATTCCT |
| Nicotiana | GATCGATTACCAAGCATAATTATCTCGTTATGGACGTAGAGGATATTCCT |
| | G C AT AC AA CATAA TAT T GT T GA GT GA GATAT CCT |
| | 710 720 730 740 750 |
| Amaranthus | AGAATTGTTAAGGAAGCTTTCTTTTTAGCTAATTCTGGTAGACCTGGACC |
| Arabidopsis | AGGATTATTGAGGAAGCTTTCTTTTTAGCTACTTCTGGTAGACCTGGACC |
| Bassia | AGAATTGTTAAGGAGGCTTTCTTTTTGGCTAATTCTGGTAGACCTGGACC |
| Brassica | AGGATCGTTCGAGAAGCTTTCTTTCTAGCTACTTCGGTTAGACCGGGACC |
| Gossypium | AGGATTGTTAGTGAGGCTTTCTTTTTAGCTTCCTCGGGCAGGCCGGGACC |
| Nicotiana | AGGGTTGTACGTGAGGCTTTTTTCCTTGCGAGATCGGGCCGGCCTGGCCC |
| | AG T T GA GCTTT TT T GC TC G G CC GG CC |
| | 760 770 780 790 800 |
| Amaranthus | TGTTTTGATTGATATTCCTAAAGATATTCAGCAACAATTGGTTGTTCCTA |
| Arabidopsis | TGTTTTGGTTGATGTTCCTAAAGATATTCAACAACAGCTTGCGATTCCTA |
| Bassia | TGTTTTGATTGATATTCCTAAGGATATTCAGCAGCAATTGGTTGTGCCTG |
| Brassica | GGTTCTTATAGACGTCCCCAAAGATGTTCAGCAACAGTTTGCGATTCCTA |
| Gossypium | TGTTCTGATTGATGTTCCTAAGGATATACAACAGCAACTTGCTGTTCCTA |
| Nicotiana | TGTTTTGATTGATGTACCTAAGGATATTCAGCAACAATTGGTGATACCTG |
| | GTT T T GA T CC AA GAT T CA CA CA T G T CCT |
| | 810 820 830 840 850 |
| Amaranthus | ACTGGGAACAGCCCATTAAATTGGGTGGGTATCTTTCTAGGTTGCCTAAA |
| Arabidopsis | ATTGGGAACAGGCTATGAGATTACCTGGTTATATGTCTAGGATGCCTAAA |
| Bassia | ATTGGGATCAGGGGGTTAGGTTAGGTGGGTATGTGTCTAGGTTGCCGAAA |
| Brassica | ACTGGGAACAGCCTATGCGCTTACCTCTTTACATGTCTACGATGCCTAAA |
| Gossypium | AATGGAACCATTCTCTTAGATTGCCAGGGTATTTGTCTAGGTTGCCTAAG |
| Nicotiana | ACTGGGATCAGCCAATGAGGTTGCCTGGTTACATGTCTAGGTTACCTAAA |
| | A TGG A CA T TT TA T TCTA G T CC AA |
| | 860 870 880 890 900 |
| Amaranthus | CCCACTTTTTCTGCTAATGAAGAGGGACTTCTTGATCAAATTGTGAGGTT |
| Arabidopsis | CCTCC------------GGAAGATTCTCATTTGGAGCAGATTGTTAGGTT |
| Bassia | TCGGTGTTTTCGGCCAATGATGAGGGGCTTCTTGAGCAGATTGTGAGGTT |
| Brassica | CCCCC------------CAAAGTTTCTCACTTAGAGCAGATTCTTAGGTT |
| Gossypium | GCTCC------------CGGTGAGGCTCATCTCGAACAGATTGTTAGATT |
| Nicotiana | TTGCC------------CAATGAGATGCTTTTAGAACAAATTGTTAGGCT |
| | G C T GA CA ATT T AG T |
| | 910 920 930 940 950 |
| Amaranthus | GGTGGGTGAGTCTAAGAGACCTGTGCTGTATACTGGAGGTGGGTGTTTGA |
| Arabidopsis | GATTTCTGAGTCTAAGAAGCCTGTGTTGTATGTTGGTGGTGGTTGTTTGA |
| Bassia | GATGAGTGAGGCTAAGAAGCCTGTGTTGTATGTGGGAGGCGGGTGTTTGA |
| Brassica | GGTTTCGGAGTCTAAGAGGCCTGTCTTGTACGTTGGAGGTGGTTGTCTGA |
| Gossypium | GGTTTCTGAGTCTAAGAAGCCTGTTTTATATGTTGGTGGTGGGTGTTTGA |
| Nicotiana | TATTTCTGAGTCAAAGAAGCCTGTTTTGTATGTGGGGGGTGGGTGTTCGC |
| | T GAG C AAGA CCTGT T TA GG GG GG TGT G |
| | 960 970 980 990 1000 |
| Amaranthus | ATTCTAGTGAAGAATTAAGGAAATTTGTCGAGTTGACAGGGATTCCCGTT |
| Arabidopsis | ATTCTAGCGATGAATTGGGTAGGTTTGTTGAGCTTACGGGGATCCCTGTT |
| Bassia | ATTCTGGGGAGGAGTTGAGGAAATTCGTCGAGTTGACTGGGATTCCGGTG |
| Brassica | ACTCGAGTGAGGAACTGCGCAGATTTGTGGAACTTACTGGCATCCCTGTT |
| Gossypium | ACTCTAGTGAGGAGTTGAAGAGGTTTGTTGAGCTTACAGGGATACCTGTT |
| Nicotiana | AATCGAGTGAGGAGTTGAGACGATTCGTGGAGCTCACCGGTATCCCCGTG |
| | A TC G GA GA T TT GT GA T AC GG AT CC GT |
| | 1010 1020 1030 1040 1050 |
| Amaranthus | GCTAGTACTTTAATGGGGTTGGGGGCTTTCCCTTGTACTGATGA---TTT |
| Arabidopsis | GCGAGTACGTTGATGGGGCTGGGATCTTATCCTTGTGATGATGA---GTT |
| Bassia | GCTAGTACTTTAATGGGTTTGGGCGCTTATCCCTGTAATGATGA---CTT |
| Brassica | GCTAGTACGTTCATGGGACTTGGATCGTATCCTTGTGACGATGAAGAGTT |
| Gossypium | GCAAGTACTTTGATGGGTCTTGGAGCCTTTCCGATTTCGGATGA---CTT |
| Nicotiana | GCAAGTACTTTGATGGGTCTTGGAGCTTTTCCAACTGGGGATGA---GCT |
| | GC AGTAC TT ATGGG T GG C T CC T GATGA T |
| | 1060 1070 1080 1090 1100 |
| Amaranthus | ATCACTTCAAATGTTGGGAATGCACGGGACTGTGTACGCGAATTACGCGG |
| Arabidopsis | GTCGTTACATATGCTTGGAATGCATGGGACTGTGTATGCAAATTACGCTG |
| Bassia | GTCTCTTCATATGTTGGGTATGCACGGGACCGTGTATGCTAATTATGCTG |
| Brassica | CTCTCTGCAAATGCTAGGAATGCATGGAACAGTGTACGCTAATTACGCTG |
| Gossypium | GTCGTTACAAATGCTTGGGATGCACGGAACTGTGTATGCCAATTATGCTG |
| Nicotiana | TTCCCTTTCAATGTTGGGTATGCATGGTACTGTTTATGCTAATTATGCTG |
| | TC T ATG T GG ATGCA GG AC GT TA GC AATTA GC G |
| | 1110 1120 1130 1140 1150 |
| Amaranthus | TGGATAAGGCTGATTTGTTGCTTGCTTTCGGGGTTAGGTTTGATGATCGA |
| Arabidopsis | TGGAGCATAGTGATTTGTTGTTGGCGTTTGGGGTAAGGTTTGATGATCGT |
| Bassia | TTGATAAGGCAGATTTGTTGCTTGCCTTTGGGGTTAGGTTTGATGATCGT |
| Brassica | TCGAGTATAGCGATCTTCTGCTTGCTTTTGGGGTTAGGTTTGACGACCGT |
| Gossypium | TTGATAAGAGTGATTTGTTGCTTGCTTTTGGAGTGAGATTTGATGATAGG |
| Nicotiana | TGGACAGTAGTGATTTATTGCTCGCATTTGGGGTGAGGTTTGATGATAGA |
| | T GA GAT T TG T GC TT GG GT AG TTTGA GA G |
| | 1160 1170 1180 1190 1200 |
| Amaranthus | GTGACTGGGAAGCTCGAGGCGTTTGCTAGCCGGGCTAAGATTGTGCACAT |
| Arabidopsis | GTCACGGGTAAGCTTGAGGCTTTTGCTAGTAGGGCTAAGATTGTTCATAT |
| Bassia | GTGACAGGGAAGCTTGAGGCGTTTGCTAGCCGGGCTAAGATCGTGCATAT |
| Brassica | GTGACCGGAAAGCTTGAGGCCTTTGCTAGCCGGGCCAAGATCGTGCATAT |
| Gossypium | GTGACGGGAAAACTTGAGGCTTTTGCCAGCCGGGCAAAGATTGTGCATAT |
| Nicotiana | GTTACTGGAAAGTTAGAAGCTTTTGCTAGCCGAGCGAAAATTGTTCACAT |
| | GT AC GG AA T GA GC TTTGC AG G GC AA AT GT CA AT |
| | 1210 1220 1230 1240 1250 |
| Amaranthus | CGATATCGATTCTGCTGAAATCGGGAAGAATAAGCAACCTCATGTGTCGA |
| Arabidapsis | TGATATTGACTCGGCTGAGATTGGGAAGAATAAGACTCCTCATGTGTCTG |
| Bassia | TGATATTGATTCTGCTGAGATTGGGAAGAATAAGCAACCCCATGTGTCAA |
| Brassica | TGATATTGATTCTACCGAAATCGGGAAGAACAAGACACCTCATGTGTCGG |
| Gossypium | CGATATCGATTCTGCCGAGATTGGGAAGAACAAGCAGCCTCATGTGTCAG |
| Nicotiana | TGATATTGATTCAGCTGAGATTGGAAAGAACAAGCAGCCTCATGTTTCCA |
| | GATAT GA TC C GA AT GG AAGAA AAG CC CATGT TC |
| | 1260 1270 1280 1290 1300 |
| Amaranthus | TTTGTGGTGAAATTAAAGTTGCATTACAGGGTTTGAATAAGATTTTGGAA |
| Arabidapsis | TGTGTGGTGATGTTAAGCTGGCTTTGCAAGGGATGAATAAGGTTCTTGAG |
| Bassia | TATGTGCTGATGTCAAGTATGCGTTGAAGGGTATGAATAAGATTTTGGAG |
| Brassica | TGTGTTGTGATGTTCAGCTAGCCTTGCAAGGGATGAACGAGGTTCTTGAG |
| Gossypium | TGTGTTCCGATGTGAAATTGGCATTGCAGGGGATAAATAAGATATTGGAG |
| Nicotiana | TTTGTGCGGATATCAAGTTGGCGTTACAGGGTTTGAATTCGATATTGGAG |
| | T TGT GA T A GC TT A GG T AA G T T GA |
| | 1310 1320 1330 1340 1350 |
| Amaranthus | TCTAGAAAAGGAAAGCTGAAATTGGATTTCTCTAATTGGAGGGAGGAGTT |
| Arabidapsis | AACCGAGCGGAGGAGCTTAAGCTTGATTTTGGAGTTTGGAGGAATGAGTT |
| Bassia | TCTAGGAAAGGGAAGTTGAAATTAAATTACTCTAGCTGGAGGGAGGAATT |
| Brassica | AACCGACGAGA------TGTGCTTGACTTCGGGGAATGGAGATGTGAATT |
| Gossypium | ACCAAGGTAGCAAAGCTGAATCTTGATTATTCGGAATGGAGGCAGGAGTT |
| Nicotiana | AGTAAGGAAGGTAAACTGAAGTTGGATTTTTCTGCTTGGAGGCAGGAGTT |
| | G T A T TGGAG GA TT |
| | 1360 1370 1380 1390 1400 |
| Amaranthus | GAATGAGCAGAAAAAGAAGTTTCCTTTGAGTTTTAAGTCTTTCGGGGACG |
| Arabidopsis | GAACGTACAGAAACAGAAGTTTCCGTTGAGCTTTAAGACGTTTGGGGAAG |
| Bassia | GGGTGAGCAAAAGAAGAAATTCCCATTGTCTTTTAAGACCTTCGGGGAAG |
| Brassica | GAACGAACAGAGACTAAAGTTCCCTCTCCGCTACAAGACGTTTGGGGAAG |
| Gossypium | AAACGAGCAGAAGCTGAAATTCCCTTTGAGTTACAAGACCTTTGGTGAAG |
| Nicotiana | GACGGTGCAGAAAGTGAAGTACCCGTTGAATTTTAAAACTTTTGGTGATG |
| | G CA A AA T CC T T AA C TT GG GA G |
| | 1410 1420 1430 1440 1450 |
| Amaranthus | CAATTCCTCCGCAATATGCCATTCAGGTTCTGGACGAGTTGACGTTGGGT |
| Arabidopsis | CTATTCCTCCACAGTATGCGATTAAGGTCCTTGATGAGTTGACTGATGGA |
| Bassia | CGATTCCTCCTCAGTATGCCATTCAGATGCTTGATGAGCTGACCAATGGT |
| Brassica | AGATTCCTCCACAGTACGCCATTCAACTACTTGACGAGCTAACCGACGGG |
| Gossypium | CTATTCCACCTCAATATGCAATTCAGGTTCTTGATGAATTAACTGGCGGG |
| Nicotiana | CTATTCCTCCGCAATATGCTATCCAGGTTCTAGATGAGTTAACTAATGGG |
| | ATTCC CC CA TA GC AT A T CT GA GA T AC GG |
| | 1460 1470 1480 1490 1500 |
| Amaranthus | GATGCGATTGTAAGTACCGGTGTTGGGCAGCACCAAATGTGGGCTGCCCA |
| Arabidopsis | AAAGCCATAATAAGTACTGGTGTCGGGCAACATCAAATGTGGGCGGCGCA |
| Bassia | AACGCTATTATTAGTACTGGTGTTGGGCAACATCAAATGTGGGCTGCTCA |
| Brassica | AAGGCAATTATCACTACTGGTGTCGGGCAACACCAGATGTGGGCCGCCCA |
| Gossypium | AATGCAATTATAAGTACTGGTGTTGGTCAGCATCAAATGTGGGCTGCTCA |
| Nicotiana | AGTGCTATTATAAGTACCGGTGTTGGGCAGCACCAGATGTGGGCTGCTCA |
| | GC AT T A TAC GGTGTYGGGCARCAYCARATGTGGGC GC CA |
| | 1510 1520 1530 1540 1550 |
| Amaranthus | ATTCTATAAGTACCGAAATCCTCGCCAATGGCTGACCTCGGGTGGTTTGG |
| Arabidopsis | GTTCTACAATTACAAGAAACCAAGGCAGTGGCTATCATCAGGAGGCCTTG |
| Bassia | GCATTACAAGTACAGAAACCCTCGCCAATGGCTGACCTCAGGTGGGTTGG |
| Brassica | ATTCTACAGATTCAAGAAACCCCGCCAATGGCTGTCTTCAGGAGGCCTAG |
| Gossypium | ATTTTACAAGTATAAGAAGCCTCGTCAATGGTTAACATCTGGGGGATTGG |
| Nicotiana | ATATTATAAGTACAGAAAGCCACGCCAATGGTTGACATCTGGTGGATTAG |
| | TA A T AA CC G CA TGG T C TC GG GG T G |
| | 1560 1570 1580 1590 1600 |
| Amaranthus | GGGCTATGGGGTTTGGTCTACCAGCCTGCTATGGAGCTGCTGTTGCTCGA |
| Arabidopsis | GAGCTATGGGATTTGGACTTCCTGCTGCGATTGGAGCGTCTGTTGCTAAC |
| Bassia | GTGCCATGGGTTTTGGTCTACCAGCCGCCATTGGAGCTGCTGTGGCTCGA |
| Brassica | GAGCCATGGGGTTCGGTCTTCCTGCAGCCATGGGAGCCGCTATAGCCAAC |
| Gossypium | GTGCTATGGGATTTGGATTGCCTGCTGCTATTGGAGCTGCCGTTGCAAAC |
| Nicotiana | GAGCGATGGGATTTGGTTTGCCCGCTGCTATTGGTGCGGCTGTTGGAAGA |
| | G GC ATGGG TT GG T CC GC GG GC C T G |
| | 1610 1620 1630 1640 1650 |
| Amaranthus | CCAGATGCGGTGGTTGTAGACATTGATGGGGATGGGAGTTTTATCATGAA |
| Arabidopsis | CCTGATGCGATAGTTGTGGATATTGACGGAGATGGAAGCTTTATAATGAA |
| Bassia | CCTGATGCAGTGGTGGTTGATATTGATGGCGATGGGAGTTTCATTATGAA |
| Brassica | CCGGGAGCAGTGGTTGTCGACATTGATGGGGATGGTAGCTTCATCATGAA |
| Gossypium | CCAGAGGCAGTCGTTGTAGACATCGATGGTGATGGAAGTTTTATCATGAA |
| Nicotiana | CCTGATGAAGTTGTGGTTGACATTGATGGTGATGGCAGTTTCATCATGAA |
| | CC G G T GT GT GA AT GA GG GATGG AG TT AT ATGAA |
| | 1660 1670 1680 1690 1700 |
| Amaranthus | TGTTCAAGAGTTGGCTACGATTAGGGTAGAGAATCTCCCGGTTAAAATCA |
| Arabidopsis | TGTGCAAGAGCTAGCCACTATTCGTGTAGAGAATCTTCCAGTGAAGGTAC |
| Bassia | TGTTCAAGAGTTGGCTACTATTAGGGTGGAAAATCTCCCTGTTAAGATAA |
| Brassica | CATTCAAGAACTGGCAACCATCAGGGTTGAGAATCTTCCAGTCAAGGTTT |
| Gossypium | CGTGCAAGAGTTGGCGACTATCCGTGTGGAAAATCTTCCGGTTAAGATAT |
| Nicotiana | TGTGCAGGAGCTAGCAACTATTAAGGTGGAGAATCTCCCAGTTAAGATTA |
| | T CA GA T GC AC AT GT GA AATCT CC GT AA T |
| | 1710 1720 1730 1740 1750 |
| Amaranthus | TGCTCTTGAACAATCAACATTTAGGTATGGTTGTTCAATTGGAAGATCGA |
| Arabidopsis | TTTTATTAAACAACCAGCATCTTGGCATGGTTATGCAATGGGAAGATCGG |
| Bassia | TGCTTTTGAATAACCAACATTTAGGTATGGTGGTTCAATTGGAAGATAGG |
| Brassica | TGCTGATTAATAATCAGCACCTCGGAATGGTCCTTCAGTGGGAAGACCAC |
| Gossypium | TATTGTTGAATAATCAGCATTTGGGTATGGTTGTTCAATGGGAGGACCGG |
| Nicotiana | TGTTACTGAATAATCAACACTTGGGAATGGTGGTTCAATGGGAGGATCGG |
| | T T T AA AA CA CA T GG ATGGT T CA T GGA GA |
| | 1760 1770 1780 1790 1800 |
| Amaranthus | TTTTACAAAGCTAACCGGGCACATACTTACCTCGGGAATCCATCCAATTC |
| Arabidopsis | TTCTACAAAGCTAACCGAGCTCACACATTTCTCGGGGATCCGGCTCAGGA |
| Bassia | TTTTATAAAGCCAATAGGGCACATACTTACCTTGGAAACCCTTCAAAAGA |
| Brassica | TTCTACGCAGCTAACAGAGCCGATTCTTTTCTGGGAGACCCGGCGAACCC |
| Gossypium | TTTTACAAGGCAAACAGGGCTCATACATACTTGGGAGACCCATCCAACGA |
| Nicotiana | TTCTATAAGGCTAACAGAGCACACACATACCTGGGGAATCCTTCTAATGA |
| | TT TA GC AA G GC A C T T GG A CC C A |
| | 1810 1820 1830 1840 1850 |
| Amaranthus | TTCCGAAATCTTCCCGGATATGCTTAAATTTGCTGAAGCATGTGATATAC |
| Arabidopsis | GGACGAGATATTCCCGAACATGTTGCTGTTTGCAGCAGCTTGCGGGATTC |
| Bassia | GTCTGAAATCTTCCCGGATATGCTTAAATTTGCTGAGGCGTGTGATATTC |
| Brassica | TGAGGCGGTATTCCCGGATATGCTGTTGTTCGCCGCATCGTGCGGTATAC |
| Gossypium | GTCGGAAATATTCCCAAATATGTTGAAATTTGCTGAAGCATGCGGGATAC |
| Nicotiana | GGCGGAGATCTTTCCTAATATGTTGAAATTTGCAGAGGCTTGTGGCGTAC |
| | G T TT CC A ATG T TT GC G C TG G T C |
| | 1860 1870 1880 1890 1900 |
| Amaranthus | CAGCAGCCCGTGTTACCAAGGTGAGCGATTTAAGGGCTGCAATTCAAACA |
| Arabidopsis | CAGCGGCGAGGGTGACAAAGAAAGCAGATCTCCGAGAAGCTATTCAGACA |
| Bassia | CTGCTGCTCGTGTCACCAAGGTTGGAGATTTGAGGGCGGCCATGCAGACA |
| Brassica | CAGCCGCCAGGGTCACCAGAAGGGAGGACCTCCGAGAGGCAATCCAGACA |
| Gossypium | CAGCTGCCCGGGTGACAAAGAAAGAAGATCTAAAAGCAGCAATGCAGAAA |
| Nicotiana | CTGCTGCGAGAGTGACACACAGGGATGATCTTAGAGCGGCTATTCAAAAG |
| | C GC GC G GT AC GA T G GC AT CA A |
| | 1910 1920 1930 1940 1950 |
| Amaranthus | ATGTTGGATACGCCAGGACCATATCTGCTGGATGTAATCGTACCACATCA |
| Arabidopsis | ATGCTGGATACACCAGGACCTTACCTGTTGGATGTGATTTGTCCGCACCA |
| Bassia | ATGTTGGATACTCCGGGACCTTACCTGCTTGATGTGATTGTACCTCATCA |
| Brassica | ATGCTGGACACACCTGGACCATTCTTGTTGGATGTGGTCTGTCCTCACCA |
| Gossypium | ATGTTGGACACTCCTGGACCTTACTTGTTGGATGTGATTGTCCCACATCA |
| Nicotiana | ATGTTAGACACTCCTGGGCCATACTTGTTGGATGTGATTGTACCTCATCA |
| | ATG T GA AC CC GG CC T TG T GATGTGATYKKMCCDCAYCA |
| | 1960 1970 1980 1990 2000 |
| Amaranthus | GGAGCATGTGCTGCCTATGATCCCTAGCGGTGCCGCCTTCAAGGACACCA |
| Arabidopsis | AGAACATGTGTTGCCGATGATCCCGAATGGTGGCACTTTCAACGATGTCA |
| Bassia | GGAGCATGTGCTGCCTATGATTCCTAGTGGTGCAGCCTTCAAGGATATCA |
| Brassica | GGACCATGTGTTACCACTCATCCCTAGTGGCGGCACCTTCAAGGACATTA |
| Gossypium | AGAACATGTCCTGCCTATGATCCCCAGTGGAGGGGCTTTCAAAGATGTGA |
| Nicotiana | GGAACATGTTCTACCTATGATTCCCAGTGGCGGGGCTTTCAAAGATGTGA |
| | RGARCATGT T CC T AT CC A GG G C TTCAA GA A |
| | 2010 2020 2030 |
| Amaranthus | TCACAGAGGGTGATGGAAGAAGGGCTTATTAG |
| Arabidopsis | TAACGGAAGGAGATGGCCGGATTAAATACTGA |
| Bassia | TTAACGAAGGTGATGGAAGAACAAGTTATTGA |
| Brassica | TTGTGTAG |
| Gossypium | TCACAGAGGGTGATGGAAGAACACAATATTGA |
| Nicotiana | TCACAGAGGGTGACGGGAGAAGTTCCTATTGA |
| | T A |

TABLE 2

Clustal W(1.4) multiple sequence alignment
Clustal W alignment of the amino acid sequences of the
tolerant (R_ALS) and five susceptible (S_80R, S_405B,
S_FS703, S_F701 and S_W501) alleles of the sunflower
ALS/AHAS. The bottom line shows the deduced consensus
sequence, conservative mutations are indicated by an
asterix (*), non-conservative mutations by a dot (.).
Pairwise Alignment Parameters:
ktup = 1 Gap Penalty = 3 Top Diagonals = 5 Window Size = 5
Multiple Alignment Parameters:
Open Gap Penalty = 10.0 Extend Gap Penalty = 0.1
Delay Divergent = 40% Gap Distance = 8
Similarity Matrix: blosum
Alignment Score = 27288; Conserved Identities = 245;
Gaps Inserted = 0

| | |
|---|---|
| | 310 320 330 340 350 |
| R_HaALS | ELTGIPVASTLMGLGAYPASSDLSLHMLGMHGTVYANYAVDKSDLLLAFG |
| S_80R | LAFG |
| S_405B | LSLHMLGMHGTVYANYAVDKSDLLLAFG |
| S_F703 | SLHMLGMHGTVYANYAVDKSDLLLAFG |
| S_FS701 | ELTGIPVASTLMGLGAYPASSDLSLHMLGMHGTVYANYAVDKSDLLLAFG |
| S_W501 | LSLHMLGMHGTVYANYAVDKSDLLLAFG |
| | LAFG |
| | 360 370 380 390 400 |
| R_HaALS | VRFDDRVTGKLEAFASRAKIVHIDIDPAEIGKNKQPHVSICGDIKVALQG |
| S_80R | VRFDDRVTGKLEAFASRAKIVHIDIDPAEIGKNKQPHVSICGDIKVALQG |
| S_405B | VRFDDRVTGKLEAFASRAKIVHIDIDPAEIGKNKQPHVSICGDIKVALQG |
| S_F703 | VRFDDRVTGKLEAFASRAKIVHIDIDPAEIGKNKQPHVSICGDIKVALQG |
| S_FS701 | VRFDDRVTGKLEAFASRAKIVHIDIDPAEIGKNKQPHVSICGDIKVALQG |
| S_W501 | VRFDDRVTGKLEAFASRAKIVHIDIDPAEIGKNKQPHVSICGDIKVALQG |
| | VRFDDRVTGKLEAFASRAKIVHIDIDPAEIGKNKQPHVSICGDIKVALQG |
| | 410 420 430 440 450 |
| R_HaALS | LNKILEEKNSVTNLDFSNWRKELDEQKVKFPLSFKTFGEAIPPQHAIQVL |
| S_80R | LNKILEEKNSVTNLDFSTWRKELDEQKMKFPLSFKTFGEAIPPQYAIQVL |
| S_405B | LNKILEEKNSVTNLDFSNWRKELDEQKVKFPLSFKTFGEAIPPQYAIQVL |
| S_F703 | LNKILEEKNSVTNLDFSTWRKELDEQKMKFPLSFKTFGEAIPPQYAIQVL |
| S_FS701 | LNKILEEKNSVTNLDFSTWRKELDEQKMKFPLSFKTFGEAIPPQYAIQVL |
| S_W501 | LNKILEEKNSVTNLDFSTWRKELDEQKMKFPLSFKTFGEAIPPQYAIQVL |
| | LNKILEEKNSVTNLDFS*WRKELDEQK*KFPLSFKTFGEAIPPQ.AIQVL |
| | 460 470 480 490 500 |
| R_HaALS | DELTGGNAIISTGVGQHQMWAAQFYKYNKPRQWLTSGGLGAMGFGLPAAI |
| S_80R | DELTGGNAIISTGVGQHQMWAAQFYKYNKPRQWLTSGGLGAMGFGLPAAI |
| S_405B | DELTGGNAIISTGVGQHQMWAAQFYKYNKPRQWLTSGGLGAMGFGLPAAI |
| S_F703 | DELTGGNAIISTGVGQHQMWAAQFYKYNKPRQWLTSGGLGAMGFGLPAAI |
| S_FS701 | DELTGGNAIISTGVGQHQMWAAQFYKYNKPRQWLTSGGLGAMGFGLPAAI |
| S_W501 | DELTGGNAIISTGVGQHQMWAAQFYKYNKPRQWLTSGGLGAMGFGLPAAI |
| | DELTGGNAIISTGVGQHQMWAAQFYKYNKPRQWLTSGGLGAMGFGLPAAI |
| | 510 520 530 540 550 |
| R_HaALS | GAAVARPDAVVVDIDGDGSFMMNVQELATIRVENLPVKILLLNNQHLGMV |
| S_80R | GAAVARPDAVVVDIDGDGSFMMNVQELATIRVENLPVKILLLNNQHLGMV |
| S_405B | GAAVARPDAVVVDIDGDGSFNMNVQELATIRVENLPVKILLLNNQHLGMV |
| S_F703 | GAAVARPDAVVVDIDGDGSFMMNVQELATIRVENLPVKILLLNNQHLGMV |
| S_FS701 | GAAVARPDAVVVDIDGDGSFMMNVQELATIRVENLPVKILLLNNQHLGMV |
| S_W501 | GAAVARPDAVVVDIDGDGSFMMNVQELATIRVENLPVKILLLNNQHLGMV |
| | GAAVARPDAVVVDIDGDGSFMMNVQELATIRVENLPVKILLLNNQHLGMV |
| | 560 570 580 590 600 |
| R_HaALS | VQWEDRFYKANRAHTYLGNPSKESEIFPNMVKFAEACDIPAARVTQKADL |
| S_80R | VQWEDRFYKANRAHTYLGNPSKESEIFPNMVKFAEACDIPAARVTQ |
| S_405B | VQWEDRFYKANRAHTYLGNPSKESEIFPNMLKFAEACDIPAARVT |
| S_F703 | VQWEDRFYKANRAHTYLGNPSKESEIFPNMVKFAEACDIPAARVTQKADL |
| S_FS701 | VQWEDRFYKANRAHTYLGNPSKESEIFPNMVKFAEACDIPAARVTQKADL |
| S_W501 | VQWEDRFYKANRAHTYLGNPSKESEIFPNMVKFAEACDIPAARVT |
| | VQWEDRFYKANRAHTYLGNPSKESEIFPNM*KFAEACDIPAARVT |
| | 610 620 630 640 |
| R_HaALS | RAAIQKMLDTPGPYLLDVIVPHQEHVLPMIPAGGGFSDVITEGDGRTKY |
| S_80R | |
| S_405B | |
| S_F703 | |
| S_FS701 | RAAIQKMLDTPGPYLLDVIVPHQEHVLPMIPAGGGFSDVITEGDGRTKY |
| S_W501 | |

TABLE 3

Clustal W(1.4) multiple sequence alignment
Clustal W alignment of the amino acid sequences of the ALS/AHAS
for 11 plants species: Amaranthus sp. (U55852), Arabidopsis
thaliana (X51514), Bassia scoparia (AF094326), Brassica napus
(X16708), Gossypium hirsutum, Nicotiana tabacum (X07645), maize
(X63553), rice (AB049822), xanthium (U16280) and sunflower (this
disclosure) including the deduced consensus sequence (bottom
line).
11 Sequences Aligned. Alignment Score = 173533
Gaps Inserted = 55 Conserved Identities = 344
Pairwise Alignment Parameters:
ktup = 1 Gap Penalty = 3 Top Diagonals = 5 Window Size = 5
Multiple Alignment Parameters:
Open Gap Penalty = 10.0 Extend Gap Penalty = 0.1
Delay Divergent = 40% Gap Distance = 8
Similarity Matrix: blosum

| | |
|---|---|
| | 10 20 30 40 50 |
| Arabidopsis | MAAATTTTTTSSSISFSTKPSPSSSKSPLPISRFSLPFSLNPNKSSSSS- |
| Oilseed rape | MASFSFFGTIPS-----SPTKASVFSLPVSVTTLPSFP--- |
| Cotton | MAAATANSALPKLSTLTSSFKSSIPISKSSLPFSTTPQKPTPYR- |
| Sunflower | MAAIHPPHPSITAK-PPPSSAAAVALPPHFAFSITSTSHKRHRL-- |
| Xanthium | MAAIPHTNPSITTK-PP-SSPPRPTFLARFTFPITSTSHKRHRL-- |
| Bassia | MASTCANPTFTP---FTSKPLKPR-SPFHSFPFPSNPKTPSSS-- |
| Amaranthus | MVTLNHISSFTK---PNKTYLQ---SSIYAIPFSNSLKPTSSSSI |
| Nicotiana | MAAAAAAPSPSFSKTLSSSSSKSS-TLLPRSTFPFPHHPHKTTPP-- |
| Maize | MATAAAASTALTGATTAAP---- |
| Rice | MATTAAAAAAALSAAATAKTGR-- |
| | * |
| | 60 70 80 90 100 |
| Arabidopsis | ---RRRGIKSSSPSSISAVLNTTTNVTTTPSPTKPTKPETFISRFAPDQP |
| Oilseed rape | ---RRR------ATRVSVSANSKKDQDRTAS--RRENPSTFSSKYAPNVP |
| Cotton | --SFDVSCSLSHASS--NPRSAATSVTPKNAP-----PHDFISRYADDEP |
| Sunflower | ----HISNVLSDSTT-------TTGATTIHP-------PPFVSRYAPDQP |
| Xanthium | ----HISNVLSDSKP-------TITHSPLPT-------KSFISRYAPDQP |
| Bassia | FRNLKITSSLSSSQP-PKPPSAVKTHSPPSPLTTDEPPQGFVSRFAPDQP |
| Amaranthus | LAALFKSHHLLLQSPKPKPPSATITQSPSS--LTDDKPSSFVSRFSPEEP |
| Nicotiana | --PLHLTPTHIHSQR--RRFTISNVISTTQKVSETQKAETFVSRFAPDEP |
| Maize | -KARRRAHLLATR-RALAAPIRCSAASPAMP---MAPPATPLRPWGPTDP |
| Rice | -KNHQRHHVLPARGRVGAAAVRCSAVSPVTPP-SPAPPATPLRPWGPAEP |
| | P |
| | 110 120 130 140 150 |
| Arabidopsis | RKGADILVEALERQGVETVFAYPGGASMEIHQALTRSSSIRNVLPRHEQG |
| Oilseed rape | RSGADILVEALERQGVDVVFAYPGGASMEIHQALTRSNTIRNVLPRHEQG |
| Cotton | RKGADILVEALVREGVKDVFAYPGGASMEIHQALTRSKIIRNVLPRHEQG |
| Sunflower | RKGADVLVEALEREGVTDVFAYPGGASMEIHQALTRSNTIRNVLPRHEQG |
| Xanthium | RKGADVLVEALEREGVTDVFAYPGGASMEIHQALTRSTTIRNVLPRHEQG |
| Bassia | RKGCDVLVEALEREGVTDVFAYPGGASMEIHQALTRSDSIRNVLPRHEQG |
| Amaranthus | RKGCDVLVEALEREGVTDVFAYPGGASMEIHQALTRSNIIRNVLPRHEQG |
| Nicotiana | RKGSDVLVEALEREGVTDVFAYPGGASMEIHQALTRSSIIRNVLPRHEQG |
| Maize | RKGADILVESLERCGVRDVFAYPGGASMEIHQALTRSPVIANHLFRHEQG |
| Rice | RKGADILVEALERCGVSDVFAYPGGASMEIHQALTRSPVITNHLFRHEQG |
| | R G*D*LVE*L R GV VFAYPGGASMEIHQALTRS I N L RHEQG |
| | 160 170 180 190 200 |
| Arabidopsis | GVFAAEGYARSSGKPGICIATSGPGATNLVSGLADALLDSVPLVAITGQV |
| Oilseed rape | GIFAAEGYARSSGKPGICIATSGPGAMNLVSGLADALFDSVPLIAITGQV |
| Cotton | GVFAAEGYARSSGIPGVCIATSGPGATNLVSGLADAMLDSIPLVAITGQV |
| Sunflower | GVFAAEGYARASGVPGVCIATSGPGATNLVSGLADALLDSVPMVAITGQV |
| Xanthium | GVFAAEGYARASGLPGVCIATSGPGATNLVSGLADALLDSVPMVAITGQV |
| Bassia | GIFAAEGYARATGRVGVCIATSGPGATNLVSGFADALLDSVPLVAITGQV |
| Amaranthus | GVFAAQGYARATGRVGVCIATSGPGATNLVSGFADALLDSVPLVAITGQV |
| Nicotiana | GVFAAEGYARATGFPGVCIATSGPGATNLVSGLADALLDSVPIVAITGQV |
| Maize | EAFAASGYARSSGRVGVCIATSGPGATNLVSALADALLDSVPMVAITGQV |
| Rice | EAFAASGYARASGRVGVCVATSGPGATNLVSALADALLDSVPMVAITGQV |
| | FAA GYAR**G G*C*ATSGPGA NLVS ADA* DS*P**AITGQV |
| | 210 220 230 240 250 |
| Arabidopsis | PRRMIGTDAFQETPIVEVTRSITKHNYLVMDVEDIPRIIEEAFFLATSGR |
| Oilseed rape | PRRMIGTMAFQETPVVEVTRTITKHNYLVMEVDDIPRIVREAFFLATSVR |
| Cotton | PRRMIGTDAFQETPIVEVTRSITKHNYLVLDVDDIPRIVSEAFFLASSGR |
| Sunflower | PRRMIGTDAFQETPIVEVTRSITKHNYLVLDVEDIPRIVREAFYLASSGR |
| Xanthium | PRRMIGTDAFQETPIVEVTRSITKHNYLVLDVEDIPRIVREAFYLASSGR |
| Bassia | PRRMIGTDAFQETPIVEVTRSITKHNYLVLDVEDIPRIVKEAFFLANSGR |
| Amaranthus | PRRMIGTDAFQETPIVEVTRSITKHNYLVLDVEDIPRIVKEAFFLANSGR |
| Nicotiana | PRRMIGTDAFQETPIVEVTRSITKHNYLVMDVEDIPRVVREAFFLARSGR |
| Maize | PRRMIGTDAFQETPIVEVTRSITKHNYLVLDVDDIPRVVQEAFFLASSGR |
| Rice | PRRMIGTDAFQETPIVEVTRSITKHNYLVLDVEDIPRVIQEAFFLASSGR |
| | PRRMIGT AFQETP*VEVTR*ITKHNYLV**V*DIPR** EAF*LA S R |
| | 260 270 280 290 300 |
| Arabidopsis | PGPVLVDVPKDIQQQLAIPNWEQAMRLPGYMSRMPK----PPEDSHLEQI |
| Oilseed rape | PGPVLIDVPKDVQQQFAIPNWEQPMRLPLYMSTMPK----PPKVSHLEQI |
| Cotton | PGPVLIDVPKDIQQQLAVPKWNHSLRLPGYLSRLPK----APGEAHLEQI |
| Sunflower | PGPVLIDVPKDIQQQLVVPKWDEPMRLPGYLSRMPK----PQYDGHLEQI |
| Xanthium | PGPVLIDVPKDIQQQLVVPKWDEPIRLPGYLSRFPK----TENNGQLEQI |
| Bassia | PGPVLIDIPKDIQQQLVVPDWDQGVRLGGYVSRLPKSVFSANDEGLLEQI |
| Amaranthus | PGPVLIDIPKDIQQQLVVPNWEQPIKLGGYLSRLPKPTFSANEEGLLDQI |
| Nicotiana | PGPVLIDVPKDIQQQLVIPDWDQPMRLPGYMSRLPK---LPN-EMLLEQI |
| Maize | PGPVLVDIPKDIQQQMAVPVWDKPMSLPGYIARLPK----PPATELLEQV |
| Rice | PGPVLVDIPKDIQQQMAVPVWDTSMNLPGYIARLPK----PPATELLEQV |
| | PGPVL*D*PKD*QQQ *P W L Y** PK L*Q* |
| | 310 320 330 340 350 |
| Arabidopsis | VRLISESKKPVLYVGGGCLNSSDELGRFVELTGIPVASTLMGLGSYPCDD |
| Oilseed rape | LRLVSESKRPVLYVGGGCLNSSEELRRFVELTGIPVASTFMGLGSYPCDD |
| Cotton | VRLVSESKKPVLYVGGGCLNSSEELKRFVELTGIPVASTLMGLGAFPISD |
| Sunflower | VRLVGEAKRPVLYVGGGCLNSDDELRRFVELTGIPVASTLMGLGAYPASS |
| Xanthium | VRLVSEAKRPVLYVGGGCLNSGDELRRFVELTGIPVASTLMGLGAYPASS |
| Bassia | VRLMSEAKKPVLYVGGGCLNSGEELRKFVELTGIPVASTLMGLGAYPCND |
| Amaranthus | VRLVGESKRPVLYTGGGCLNSSEELRKFVELTGIPVASTLMGLGAFPCTD |
| Nicotiana | VRLISESKKPVLYVGGGCSQSSEELRRFVELTGIPVASTLMGLGAFPTGD |
| Maize | LRLVGESRRPVLYVGGGCAASGEELRRFVELTGIPVTTTLMGLGNFPSDD |
| Rice | LRLVGESRRPILYVGGGCSASGDELRWFVELTGIPVTTTLMGLGNFPSDD |
| | *RL* E***P*LY GGGC S *EL FVELTGIPV**T MGLG *P |
| | 360 370 380 390 400 |
| Arabidopsis | -ELSLHMLGMHGTVYANYAVEHSDLLLAFGVRFDDRVTGKLEAFASRAKI |
| Oilseed rape | EEFSLQMLGMHGTVYANYAVEYSDLLLAFGVRFDDRVTGKLEAFASRAKI |
| Cotton | -DLSLQMLGMHGTVYANYAVDKSDLLLAFGVRFDDRVTGKLEAFASRAKI |
| Sunflower | -DLSLHMLGMHGTVYANYAVDKSDLLLAFGVRFDDRVTGKLEAFASRAKI |
| Xanthium | -DLSLHMLGMHGTVYANYAVDKSDLLLAFGVRFDDRVTGKLEAFASRAKI |
| Bassia | -DLSLHMLGMHGTVYANYAVDKADLLLAFGVRFDDRVTGKLEAFASRAKI |
| Amaranthus | -DLSLQMLGMHGTVYANYAVDKADLLLAFGVRFDDRVTGKLEAFASRAKI |
| Nicotiana | -ELSLSMLGMHGTVYANYAVDSSDLLLAFGVRFDDRVTGKLEAFASRAKI |
| Maize | -PLSLRMLGMHGTVYANYAVDKADLLLALGVRFDDRVTGKIEAFASRAKI |
| Rice | -PLSLRMLGMHGTVYANYAVDKADLLLAFGVRFDDRVTGKIEAFASRAKI |
| | SL MLGMHGTVYANYAV* *DLLLA GVRFDDRVTGK*EAFASRAKI |
| | 410 420 430 440 450 |
| Arabidopsis | VHIDIDSAEIGKNKTPHVSVCGDVKLALQGMNKVLENRAEELKLDFGVWR |
| Oilseed rape | VHIDIDSTEIGKNKTPHVSVCCDVQLALQGMNEVLENRRD--VLDFGEWR |
| Cotton | VHIDIDSAEIGKNKQPHVSVCSDVKLALQGINKILETKVAKLNLDYSEWR |
| Sunflower | VHIDIDPAEIGKNKQPHVSICGDIKVALQGLNKILEEKNSVTNLDFSTWR |
| Xanthium | VHIDIDSAEIGKNKQPHVSICGDIKVALQGLNKILEVKNSVTNLDFSNWR |
| Bassia | VHIDIDSAEIGKNKQPHVSICADVKYALKGMNKILESRKGKLKLNYSSWR |
| Amaranthus | VHIDIDSAEIGKNKQPHVSICGEIKVALQGLNKILESRKGKLKLDFSNWR |
| Nicotiana | VHIDIDSAEIGKNKQPHVSICADIKLALQGLNSILESKEGKLKLDFSAWR |
| Maize | VHVDIDPAEIGKNKQPHVSICADVKLALQGMNALLEGSTSKKSFDFGSWN |
| Rice | VHIDIDPAEIGKNKQPHVSICADVKLALQGLNALLQQSTTKTSSDFSAWH |
| | VH*DID *EIGKNK PHVS*C* *** AL*G*N *L* * W |
| | 460 470 480 490 500 |
| Arabidopsis | NELNVQKQKFPLSFKTFGEAIPPQYAIKVLDELTDGKAIISTGVGQHQMW |
| Oilseed rape | CELNEQRLKFPLRYKTFGEEIPPQYAIQLLDELTDGKAIITTGVGQHQMW |
| Cotton | QELNEQKLKFPLSYKTFGEAIPPQYAIQVLDELTGGNAIISTGVGQHQMW |
| Sunflower | KELDEQKMKFPLSFKTFGEAIPPQYAIQVLDELTGGNAIISTGVGQHQMW |
| Xanthium | KELDEQKVKYPLSFKTFGEAIPPQYAIQVLDELTGGNAIISTGVGQHQMW |
| Bassia | EELGEQKKKFPLSFKTFGEAIPPQYAIQMLDELTNGNAIISTGVGQHQMW |
| Amaranthus | EELNEQKKKFPLSFKSFGDAIPPQYAIQVLDELTLGDAIVSTGVGQHQMW |
| Nicotiana | QELTVQKVKYPLNFKTFGDAIPPQYAIQVLDELTNGSAIISTGVGQHQMW |
| Maize | DELDQQKREFPLGYKTSNEEIQPQYAIQVLDELTKGEAIIGTGVGQHQMW |
| Rice | NELDQQKREFPLGYKTFGEEIPPQYAIQVLDELTKGEAIIATGVGQHQMW |
| | EL Q* *PL *K* ** I PQYAI**LDELT G AI* TGVGQHQMW |
| | 510 520 530 540 550 |
| Arabidopsis | AAQFYNYKKPRQWLSSGGLGAMGFGLPAAIGASVANPDAIVVDIDGDGSF |
| Oilseed rape | AAQFYRFKKPRQWLSSGGLGAMGFGLPAAMGAAIANPGAVVVDIDGDGSF |
| Cotton | AAQFYKYKKPRQWLTSGGLGAMGFGLPAAIGAAVANPEAVVVDIDGDGSF |
| Sunflower | AAQFYKYNKPRQWLTSGGLGAMGFGLPAAIGAAVARPDAVVVDIDGDGSF |
| Xanthium | AAQFYKYNKPRQWLTSGGLGAMGFGLPAAIGAAVARPDAVVVDIDGDGSF |
| Bassia | AAQHYKYRNPRQWLTSGGLGAMGFGLPAAIGAAVARPDAVVVDIDGDGSF |
| Amaranthus | AAQFYKYRNPRQWLTSGGLGAMGFGLPACYGAAVARPDAVVVDIDGDGSF |
| Nicotiana | AAQYYKYRKPRQWLTSGGLGAMGFGLPAAIGAAVGRPDEVVVDIDGDGSF |
| Maize | AAQYYTYKRPRQWLSSAGLGAMGFGLPAAAGASVANPGVTVVDIDGDGSF |
| Rice | AAQYYTYKRPRQWLSSAGLGAMGFGLPAAAGASVANPGVTVVDIDGDGSF |
| | AAQ Y * PRQWL*S GLGAMGFGLPA* GA** P VVDIDGDGSF |
| | 560 570 580 590 600 |
| Arabidopsis | IMNVQELATIRVENLPVKVLLLNNQHLGMVMQWEDRFYKANRAHTFLGDP |
| Oilseed rape | IMNIQELATIRVENLPVKVLLINNQHLGMVLQWEDHFYAANRADSFLGDP |
| Cotton | IMNVQELATIRVENLPVKILLLNNQHLGMVVQWEDRFYKANRAHTYLGDP |
| Sunflower | NMNVQELATIRVENLPVKILLLNNQHLGMVVQWEDRFYKANRAHTYLGNP |
| Xanthium | IMNVQELATIRVENLPVKILLLNNQHLGMVVQWEDRFYKANRAHTYLGNP |
| Bassia | IMNVQELATIRVENLPVKIMLLNNQHLGMVVQLEDRFYKANRAHTYLGNP |
| Amaranthus | IMNVQELATIRVENLPVKIMLLNNQHLGMVVQLEDRFYKANRAHTYLGNP |
| Nicotiana | IMNVQELATIKVENLPVKIMLLNNQHLGMVVQWEDRFYKANRAHTYLGNP |
| Maize | LMNVQELAMIRIENLPVKVFVLNNQHLGMVVQWEDRFYKANRAHTYLGNP |
| Rice | LMNIQELALIRIENLPVKVMVLNNQHLGMVVQWEDRFYKANRAHTYLGNP |
| | *MN*QELA I**ENLPVK* **NNQHLGMV*Q ED*FY ANRA **LG P |
| | 610 620 630 640 650 |
| Arabidopsis | AQEDEIFPNMLLFAAACGIPAARVTKKADLREAIQTMLDTPGPYLLDVIC |
| Oilseed rape | ANPEAVFPDMLLFAASCGIPAARVTRREDLREAIQTMLDTPGPFLLDVVC |
| Cotton | SNESEIFPNMLKFAEACGIPAARVTKKEDLKAAMQKMLDTPGPYLLDVIV |
| Sunflower | SKESEIFPNMVKFAEACDIPAARVTQKADLRAAIQKMLDTPGPYLLDVIV |
| Xanthium | SKESEIFPNMLKFAEACDIPAARVTRKADLRAAIQKMLDTPGPYLLDVIV |
| Bassia | SKESEIFPDMLKFAEACDIPAARVTKVGDLRAAMQTMLDTPGPYLLDVIV |
| Amaranthus | SNSSEIFPDMLKFAEACDIPAARVTKVSDLRAAIQTMLDTPGPYLLDVIV |
| Nicotiana | SNEAEIFPNMLKFAEACGVPAARVTHRDDLRAAIQKMLDTPGPYLLDVIV |
| Maize | ENESEIYPDFVTIAKGFNIPAVRVTKKNEVRAAIKKMLETPGPYLLDIIV |
| Rice | ECESEIYPDFVTIAKGFNIPAVRVTKKSEVRAAIKKMLETPGPYLLDIIV |
| | **P * A *P* RV* *** A** ML*TPGP*LLD** |
| | 660 670 |
| Arabidopsis | PHQEHVLPMIPNGGTFNDVITEGDGRIKY |
| Oilseed rape | PHQDHVLPLIPSGGTFKDIIV |
| Cotton | PHQEHVLPMIPSGGAFKDVITEGDGRTQY |
| Sunflower | PHQEHVLPMIPAGGGFSDVITEGDGRTKY |
| Xanthium | PHQEHVLPMIPAGGGFMDVITEGDGRMKY |
| Bassia | PHQEHVLPMIPSGAAFKDIINEGDGRTSY |
| Amaranthus | PHQEHVLPMIPSGAAFKDTITEGDGRRAY |
| Nicotiana | PHQEHVLPMIPSGGAFKDVITEGDGRSSY |
| Maize | PHQEHVLPMIPSGGAFKDMILDGDGRTVY |
| Rice | PHQEHVLPMIPSGCAFKDMILDGDGRTVY |
| | PEQ*HVLP*IP G F D I |

1-47. (canceled)

48. An isolated DNA molecule comprising about 15 successive nucleotides of the nucleotide sequence set forth in SEQ ID NO:1, wherein said DNA molecule comprises a nucleotide polymorphism, wherein said polymorphism allows to differentiate between an allele conferring tolerance to an inhibitor of ALS/AHAS activity and an allele sensitive to said inhibitor in a sunflower plant, wherein said polymorphism is at a position corresponding to any one of positions 1566, 1229, 1232 or 1235 in SEQ ID NO:1.

49. The DNA molecule according to claim 48, wherein said polymorphism allows to differentiate between a nucleotide sequence encoding a imidazolinone-sensitive ALS/AHAS and a nucleotide sequence encoding a imidazolinone-tolerant ALS/AHAS in a sunflower plant.

50. The DNA molecule according to claim 48, wherein DNA molecule comprises about 20 successive nucleotides of the nucleotide sequence set forth in SEQ ID NO:1.

51. The DNA molecule according to claim 48, wherein said DNA molecule is an amplified PCR fragment.

52. An oligonucleotide capable of hybridizing to a DNA molecule comprising a nucleotide sequence set forth in SEQ ID NO:1, wherein said oligonucleotide comprises a nucleotide corresponding to any one of positions 1566, 1229, 1232 or 1235 of SEQ ID NO:1 or a complement thereto.

53. The oligonucleotide according to claim 52, wherein said oligonucleotide further comprises a detectable label.

54. A method of identifying a plant tolerant to an inhibitor of ALS/AHAS activity comprising the steps of:
a) obtaining a sample from a plant;
b) detecting in said sample a DNA molecule according to claim 48, the presence of said DNA molecule being indicative of an allele conferring tolerance to an inhibitor of ALS/AHAS activity in said plant,
wherein said plant is tolerant to an inhibitor of ALS/AHAS activity.

55. A method of selecting a plant tolerant to an inhibitor of ALS/AHAS activity from a population of plants comprising the steps of:
a) providing a population of plants;
b) obtaining a sample of a plant of said population;
c) detecting in said sample a DNA molecule according to claim 48, the presence of said DNA molecule being indicative of an allele conferring tolerance to an inhibitor of ALS/AHAS activity in said plant;
d) selecting said plant, wherein said plant is tolerant to an inhibitor of ALS/AHAS activity.

56. A method for introgressing tolerance to an inhibitor of ALS/AHAS activity into a plant comprising the steps of:
a) obtaining a plant tolerant to inhibitor of ALS/AHAS activity;
b) crossing said plant of step a) with a plant which sensitive or less tolerant to said inhibitor;
c) detecting in a plant resulting from the cross in step b) a DNA molecule according to claim 48, the presence of said DNA molecule being indicative of an allele conferring tolerance to an inhibitor of ALS/AHAS activity in said plant;
d) selecting a plant of step c) for further breeding, wherein said plant is tolerant to an inhibitor of ALS/AHAS activity.

57. The method according to claim 56, further comprising repeating steps b) to d) until the tolerance to said inhibitor is introgressed into said plant which sensitive or less tolerant to said inhibitor.

58. A method to determine whether a plant is homozygous or heterozygous for an allele conferring tolerance to an inhibitor of ALS/AHAS activity comprising:
a) obtaining a sample of a plant;
b) detecting in said sample a DNA molecule according to claim 48, wherein said step of detecting is carried out using a co-dominant marker;
c) determining whether a nucleotide sequence encoding an A protein having ALS/AHAS activity tolerant to an inhibitor of ALS/AHAS activity is heterozygous or homozygous in said plant.

59. The method according to claim 54, wherein said inhibitor of ALS/AHAS activity is an imidazolinone herbicide.

60. The method according to any one of claims 54, wherein said plant is sunflower.

61. A kit for detecting a single nucleotide polymorphism indicative for tolerance or sensitivity to an inhibitor of ALS/AHAS activity in a sunflower plant comprising an oligonucleotide according to claim 52.

62. The kit according to claim 61, wherein said oligonucleotide is any one of the oligonucleotides set forth in SEQ ID NO:8 or SEQ ID NO:9, SEQ ID NO:10, SEQ ID NO:12, SEQ ID NO:11 or SEQ ID NO:13, SEQ ID NO:14, SEQ ID NO:15, SEQ ID NO:16 or SEQ ID NO:17.


SEQUENCE LISTING (17 sequences)

SEQ ID NO: 1 - DNA, 2938 bp, Helianthus annuus
  CDS (234)..(2180)
tggtttatgaagaagtatagattttcccacccaaaatgaaggttcactaaacaaatggttgccatgtcatcaaagtggcttcaccattcaaagacaaatagcccaaatcatctcctttattaatatatatagattgtaataatttaaaacaaacacaaccacatgtttgaatcactggactccttttataaccataatacaagcaaataccaaccctaaacagacattcaacaatggccgccatacatcctccccacccttccatcaccgccaaaccaccgccatcttccgccgccgccgtcgtactaccaccccacttcgcattctccatcacctccacttcccacaaacgccaccgcctccacatctccaacgtcctctccgactccaccaccaccaccggtgccaccaccattcacccaccgccattcgtctcccggtacgcgccagatcaaccgagaaaaggcgcagacgtgctcgtggaagctctagaacgggaaggtgtcaccgacgtcttcgcctaccccggcggcgcgtcaatggagatccacctagctctcacgcgctcaaacaccatccgcaatgtcctcccccgtcacgaacagggcggcgtgttcgccgcagaaggctacgcacgcgcctccggtcttcccggcgtgtgtatcgccacttccggtcccggagctacgaacctagttagtggtcttgctggcgcgttgttagacagtgtccccatggtggcaatcaccggtcaagttccccggagaatgatcggaaccgatgtgtttcaagaaaccccaattgttgaggtaacacgttcgattactaaacataattatcttgtgttggatgttgaggatattcccagaattgttcgtgaggctttttatcttgcgagttcgggtcgacccggcccggttttgatagatgtaccgaaagatatacagcaacagttagtggtgccaaaatgggatgaaccgatgaggttaccgggttatttgtctagaatgccaaagcctcaatatgatggccatttggaacagattgttaggttggtgggggaagcgaaaaggccggttttgtatgtgggtggtgggtgtttgaattcggatgatgagttgaggcggtttgtggagcttacggggattccggttgcaagtactttgatggggctcggagcgtaccctgcttcgagtgatttgtcgcttcatatgcttgggatgcatggtacggtttatgcgaattatgcggttgataagagtgatttgttgcttgcgtttggggtgcggtttgatgaccgtgtgacggggaagcttgaggcgtttgctagtagggcgaagattgttcatattgatattgatccggctgaaattgggaagaataaacagccgcatgtgtcgatttgtggggatattaaggtcgcgttacagggtttgaacaagattttggaggaaaagaattcggtgactaatcttgatttttcgaactggagaaaggaattggatgaacaaaaagtgaagtttccgttgagctttaaaacgtttggcgaagcgattcctccacagtatgctattcaagttcttgatgagttaacgggcgggaatgcaattattagcaccggtgtcgggcaacatcagatgtgggctgctcagttttacaaatacaacaaacctagacaatggctgacgtctggcgggctaggggcaatgggttttgggctgcccgctgctatcggggcggccgttgcaagacctgatgcggtagtagttgacatcgacggtgacggaagctttatgatgaatgttcaagagttagccacaatccgtgttgaaaatctgccggttaagattttattacttaataatcagcatttgggtatggtggttcagtgggaggatcggttttacaaggcgaatagggctcatacctacttaggaaacccgtcaaaagagtcggaaatattccctaacatggtgaagtttgctgaagcctgtgatatcccggctgctcgagtgacccaaaaggcggatctacgagcagctattcagaagatgttggatacacccgggccttacttgttggatgtgattgtgccgcatcaagaacacgtgttgcccatgatcccggctggcggaggtttctcggatgtgatcaccgagggtgatggcagaacgaaatattgaggtgtccaaagtcgcgggttgtgtaagcagtttgtcggttttgaagttaatgttttgttgtgtaatttagtttgaatgttctggatcagttatcaatgtttcttttaattaatagctctcgcgaataaatttccaagttttatcttttgctttatgtttatattgaagacaaacggtatgtttatattagcgagtgataatgttctctaaggtttatatcttttgctaatgttctataattccaaaagaataataattggttgaaaatgcattttgttggttcttgaattgttaagtgttcagtcgtctctttctgtccggttgtaaagtgcattttgtggtcccttgctcgctgaaacatatcgacagaaataatatattaaaaaccacatattaaagcataaaagcaaaccagaatcatggcaagtgttttctgtagaggtggcaagatgattgggtcgagtgggtatggtaacgggtcaaagtttcggttgaaacagatcatttgacccgcaaaaggcaaatgagaaatctggaggctttgtcaataggattgttgttacatgccaaggcgttggatctcgtttaggtaacttaattagcctccatataacatcggtttgtttgtaaaaaaatatacgattaatgactataatataaacgatgaaatacagaaatgttaaccgagcttgcgagaagagaaaaggaagcagggattattccagaagctgaagttggccttttcatgaaggcga

SEQ ID NO: 2 - protein, 649 aa, Helianthus annuus
MAAIHPPHPSITAKPPPSSAAAVVLPPHFAFSITSTSHKRHRLHISNVLSDSTTTTGATTIHPPPFVSRYAPDQPRKGADVLVEALEREGVTDVFAYPGGASMEIHLALTRSNTIRNVLPRHEQGGVFAAEGYARASGLPGVCIATSGPGATNLVSGLAGALLDSVPMVAITGQVPRRMIGTDVFQETPIVEVTRSITKHNYLVLDVEDIPRIVREAFYLASSGRPGPVLIDVPKDIQQQLVVPKWDEPMRLPGYLSRMPKPQYDGHLEQIVRLVGEAKRPVLYVGGGCLNSDDELRRFVELTGIPVASTLMGLGAYPASSDLSLHMLGMHGTVYANYAVDKSDLLLAFGVRFDDRVTGKLEAFASRAKIVHIDIDPAEIGKNKQPHVSICGDIKVALQGLNKILEEKNSVTNLDFSNWRKELDEQKVKFPLSFKTFGEAIPPQYAIQVLDELTGGNAIISTGVGQHQMWAAQFYKYNKPRQWLTSGGLGAMGFGLPAAIGAAVARPDAVVVDIDGDGSFMMNVQELATIRVENLPVKILLLNNQHLGMVVQWEDRFYKANRAHTYLGNPSKESEIFPNMVKFAEACDIPAARVTQKADLRAAIQKMLDTPGPYLLDVIVPHQEHVLPMIPAGGGFSDVITEGDGRTKY

SEQ ID NO: 3 - DNA, 2938 bp, Helianthus annuus
  CDS (234)..(2180)
  variation (1229): polymorphism
tggtttatgaagaagtatagattttcccacccaaaatgaaggttcactaaacaaatggttgccatgtcatcaaagtggcttcaccattcaaagacaaatagcccaaatcatctcctttattaatatatatagattgtaataatttaaaacaaacacaaccacatgtttgaatcactggactccttttataaccataatacaagcaaataccaaccctaaacagacattcaacaatggccgccatacatcctccccacccttccatcaccgccaaaccaccgccatcttccgccgccgccgtcgtactaccaccccacttcgcattctccatcacctccacttcccacaaacgccaccgcctccacatctccaacgtcctctccgactccaccaccaccaccggtgccaccaccattcacccaccgccattcgtctcccggtacgcgccagatcaaccgagaaaaggcgcagacgtgctcgtggaagctctagaacgggaaggtgtcaccgacgtcttcgcctaccccggcggcgcgtcaatggagatccacctagctctcacgcgctcaaacaccatccgcaatgtcctcccccgtcacgaacagggcggcgtgttcgccgcagaaggctacgcacgcgcctccggtcttcccggcgtgtgtatcgccacttccggtcccggagctacgaacctagttagtggtcttgctggcgcgttgttagacagtgtccccatggtggcaatcaccggtcaagttccccggagaatgatcggaaccgatgtgtttcaagaaaccccaattgttgaggtaacacgttcgattactaaacataattatcttgtgttggatgttgaggatattcccagaattgttcgtgaggctttttatcttgcgagttcgggtcgacccggcccggttttgatagatgtaccgaaagatatacagcaacagttagtggtgccaaaatgggatgaaccgatgaggttaccgggttatttgtctagaatgccaaagcctcaatatgatggccatttggaacagattgttaggttggtgggggaagcgaaaaggccggttttgtatgtgggtggtgggtgtttgaattcggatgatgagttgaggcggtttgtggagcttacggggattccggttgcaagtactttgatggggctcggagcgtaccctgcttcgagtgatttgtcgcttcatatgcttgggatgcatgggactgtctatgcgaattatgcggttgataagagtgatttgttgcttgcgtttggggtgcggtttgatgaccgtgtgacggggaagcttgaggcgtttgctagtagggcgaagattgttcatattgatattgatccggctgaaattgggaagaataaacagccgcatgtgtcgatttgtggggatattaaggtcgcgttacagggtttgaacaagattttggaggaaaagaattcggtgactaatcttgatttttcgaactggagaaaggaattggatgaacaaaaagtgaagtttccgttgagctttaaaacgtttggcgaagcgattcctccacagcatgctattcaagttcttgatgagttaacgggcgggaatgcaattattagcaccggtgtcgggcaacatcagatgtgggctgctcagttttacaaatacaacaaacctagacaatggctgacgtctggcgggctaggggcaatgggttttgggctgcccgctgctatcggggcggccgttgcaagacctgatgcggtagtagttgacatcgacggtgacggaagctttatgatgaatgttcaagagttagccacaatccgtgttgaaaatctgccggttaagattttattacttaataatcagcatttgggtatggtggttcagtgggaggatcggttttacaaggcgaatagggctcatacctacttaggaaacccgtcaaaagagtcggaaatattccctaacatggtgaagtttgctgaagcctgtgatatcccggctgctcgagtgacccaaaaggcggatctacgagcagctattcagaagatgttggatacacccgggccttacttgttggatgtgattgtgccgcatcaagaacacgtgttgcccatgatcccggctggcggaggtttctcggatgtgatcaccgagggtgatggcagaacgaaatattgaggtgtccaaagtcgcgggttgtgtaagcagtttgtcggttttgaagttaatgttttgttgtgtaatttagtttgaatgttctggatcagttatcaatgtttcttttaattaatagctctcgcgaataaatttccaagttttatcttttgctttatgtttatattgaagacaaacggtatgtttatattagcgagtgataatgttctctaaggtttatatcttttgctaatgttctataattccaaaagaataataattggttgaaaatgcattttgttggttcttgaattgttaagtgttcagtcgtctctttctgtccggttgtaaagtgcattttgtggtcccttgctcgctgaaacatatcgacagaaataatatattaaaaaccacatattaaagcataaaagcaaaccagaatcatggcaagtgttttctgtagaggtggcaagatgattgggtcgagtgggtatggtaacgggtcaaagtttcggttgaaacagatcatttgacccgcaaaaggcaaatgagaaatctggaggctttgtcaataggattgttgttacatgccaaggcgttggatctcgtttaggtaacttaattagcctccatataacatcggtttgtttgtaaaaaaatatacgattaatgactataatataaacgatgaaatacagaaatgttaaccgagcttgcgagaagagaaaaggaagcagggattattccagaagctgaagttggccttttcatgaaggcga

SEQ ID NO: 4 - protein, 649 aa, Helianthus annuus
MAAIHPPHPSITAKPPPSSAAAVVLPPHFAFSITSTSHKRHRLHISNVLSDSTTTTGATTIHPPPFVSRYAPDQPRKGADVLVEALEREGVTDVFAYPGGASMEIHLALTRSNTIRNVLPRHEQGGVFAAEGYARASGLPGVCIATSGPGATNLVSGLAGALLDSVPMVAITGQVPRRMIGTDVFQETPIVEVTRSITKHNYLVLDVEDIPRIVREAFYLASSGRPGPVLIDVPKDIQQQLVVPKWDEPMRLPGYLSRMPKPQYDGHLEQIVRLVGEAKRPVLYVGGGCLNSDDELRRFVELTGIPVASTLMGLGAYPASSDLSLHMLGMHGTVYANYAVDKSDLLLAFGVRFDDRVTGKLEAFASRAKIVHIDIDPAEIGKNKQPHVSICGDIKVALQGLNKILEEKNSVTNLDFSNWRKELDEQKVKFPLSFKTFGEAIPPQHAIQVLDELTGGNAIISTGVGQHQMWAAQFYKYNKPRQWLTSGGLGAMGFGLPAAIGAAVARPDAVVVDIDGDGSFMMNVQELATIRVENLPVKILLLNNQHLGMVVQWEDRFYKANRAHTYLGNPSKESEIFPNMVKFAEACDIPAARVTQKADLRAAIQKMLDTPGPYLLDVIVPHQEHVLPMIPAGGGFSDVITEGDGRTKY

SEQ ID NO: 5 - DNA, 20 bp, Artificial Sequence
  Description of Artificial Sequence oligonucleotide
gtnttygcbtacccwgghgg

SEQ ID NO: 6 - DNA, 26 bp, Artificial Sequence
  Description of Artificial Sequence oligonucleotide
gcccacatytgrtgytgcccracacc

SEQ ID NO: 7 - DNA, 29 bp, Artificial Sequence
  Description of Artificial Sequence oligonucleotide
acatgytcytgrtghggdmmratcacatc

SEQ ID NO: 8 - DNA, 23 bp, Artificial Sequence
  Description of Artificial Sequence oligonucleotide
gaggctttttatcttgcgagttc

SEQ ID NO: 9 - DNA, 23 bp, Artificial Sequence
  Description of Artificial Sequence oligonucleotide
ccttagagaacattatcactcgc

SEQ ID NO: 10 - DNA, 17 bp, Artificial Sequence
  Description of Artificial Sequence oligonucleotide
ggatgcatgggactgtc

SEQ ID NO: 11 - DNA, 17 bp, Artificial Sequence
  Description of Artificial Sequence oligonucleotide
ggatgcatggtacggtt

SEQ ID NO: 12 - DNA, 21 bp, Artificial Sequence
  Description of Artificial Sequence oligonucleotide
ccgaaactttgacccgttacc

SEQ ID NO: 13 - DNA, 23 bp, Artificial Sequence
  Description of Artificial Sequence oligonucleotide
ccttagagaacattatcactcgc

SEQ ID NO: 14 - DNA, 20 bp, Artificial Sequence
  Description of Artificial Sequence oligonucleotide
cttgaatagcatactgtgga

SEQ ID NO: 15 - DNA, 19 bp, Artificial Sequence
  Description of Artificial Sequence oligonucleotide
ttgaatagcatgctgtgga

SEQ ID NO: 16 - DNA, 19 bp, Artificial Sequence
  Description of Artificial Sequence oligonucleotide
cattcccgcccgttaactc

SEQ ID NO: 17 - DNA, 19 bp, Artificial Sequence
  Description of Artificial Sequence oligonucleotide
gtttggcgaagcgattcct